(19) United States
(12) Patent Application Publication
(10) Pub. No.: US 2011/0288057 A1
(43) Pub. Date: Nov. 24, 2011

(54) SALICYLIC ACID DERIVATIVES BEING FARNESYL PYROPHOSPHATE SYNTHASE ACTIVITY INHIBITORS

(75) Inventors: Simona Cotesta, Basel (CH); Joseph Fraser Glickman, Cambridge, MA (US); Wolfgang Jahnke, Basel (CH); Andreas Marzinzik, Basel (CH); Silvio Ofner, Basel (CH); Jean-Michel Rondeau, Basel (CH); Thomas Zoller, Basel (CH)

(73) Assignee: Novartis AG, Basel (CH)

(21) Appl. No.: 13/123,681

(22) PCT Filed: Oct. 12, 2009
(86) PCT No.: PCT/EP2009/063257

§ 371 Date: Apr. 11, 2011

(30) Foreign Application Priority Data
Oct. 13, 2008 (EP) 08166473.2

(51) Int. Cl. A61K 31/606; C07C 229/64; C07D 207/26; C07D 295/155; A61P 3/00; C07D 295/185; C07D 243/08; A61K 31/603; A61K 31/625; A61P 35/00; C07C 65/24; C07D 223/04

(52) U.S. Cl. 514/166; 562/466; 562/453; 548/551; 546/239; 540/610; 544/391; 540/575; 544/172; 514/159

(57) ABSTRACT

The invention relates to the use of and mainly novel compounds of the formula I

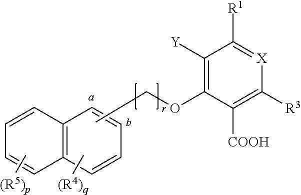

wherein the moieties are as defined in the description, which are useful as farnesyl pyrophosphate synthase modulators and e.g. in the treatment of proliferative diseases.

### SUMMARY OF THE INVENTION

The invention relates to the salicylic acid derivatives for use in the treatment of a disorder that depends on the activity of farnesyl pyrophosphate synthase (FPPS), especially a proliferative disease and/or a cholesterol biosynthesis related disorder, the use of said salicylic acid derivatives in the treatment, or for the manufacture of a pharmaceutical preparation that is useful in the treatment, of a disorder mentioned above or especially below, a method of treatment of a disorder mentioned above or especially below comprising administering a salicylic acid derivative to a warm-blooded animal, especially human, a pharmaceutical preparation for the treatment of a disorder mentioned above or especially below, a method for the manufacture of such a pharmaceutical preparation, novel salicylic acid derivatives, these compounds for use in the treatment of a disorder of a warm-blooded animal, especially a human, preferably a disorder mentioned above or especially below, a pharmaceutical preparation comprising such a compound and at least one pharmaceutically acceptable carrier material, a process or method of manufacture of these novel compounds and methods comprising the administration and uses of them as mentioned above and below.

### BACKGROUND OF THE INVENTION

Zometa is a bisphosphonate drug which is currently used for osteoporosis and metastatic bone cancers. Additionally it has anti-parasitic activity in vitro.

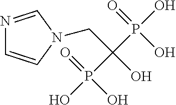

Recently, it was found that Zometa, originally discovered in the absence of knowledge about the drug target, is a potent and selective Farnesyl pyrophosphate synthase (FPPS) inhibitor.

FPPS is a key branchpoint enzyme in the mevalonate pathway. The enzyme catalyzes the head-to-tail-(1′-4)-condensation of dimethylallyl pyrophosphate (DMAPP) and of isopentenyl pyrophosphate (IPP) to geranyl pyrophosphate (GPP) and a second head-to-tail condensation of GPP with IPP to farnesyl pyrophosphate (FPP). The mechanism of these condensation reactions is interesting in that FPPS belongs to the few enzymes that catalyse a reaction in which a carbocation is formed as intermediate. FPP is, on the one hand, a precursor of steroid, especially cholesterol synthesis. Therefore inhibition of this enzyme leads to lowered cholesterol synthesis and thus lowered cholesterol levels in blood (J. F. Reilly et al., Biochem. J. (2002) 366 (501-510)).

On the other hand, protein prenyltransferases catalyze the transfer of the carbon moiety of C15 farnesyl pyrophosphate or geranylgeranyl pyrophosphate synthase to a conserved cysteine residue in a CaaX motif of protein and peptide substrates. The addition of a farnesyl group is required to anchor proteins to the cell membrane. For example, many regulatory G proteins are anchored to cell membranes by such farnesylation. Recent data suggest that geranyl-geranylation, especially of Rho GTPases, may be the main target of their anti-invasive effect although their apoptotic effect may be related to the inhibition of Ras farnesylation. Inhibition of farnesylation by inhibition of FPPS is therefore to be regarded as useful in the treatment of various proliferative diseases such as cancer and tumor diseases where dysregulation of G proteins is involved, for example in the treatment of prostate tumoral cells, and an anti-tumor effect of alendronate, zoledronate and pamidronate was correlated to their inhibition of the mevalonate pathway in prostate cells, and antitumor effects were examined (see e.g. M. Goffinet et al., BMC Cancer 2006, 6:60). A direct anti-tumor potential of zoledronate has been observed in various animal models (summarized in Croucher P. et al., The Breast 2003, Suppl. 2:S30). Further, for example, the binding of FPPS to FGF receptors (FGFRs) could be demonstrated, and it was shown that over-expression of FPPS in fibroblasts also promotes increased farnesylation of Ras, and temporally extends FGF-2-stimulated activation of the Ras/ERK (extracellular-signal-regulated kinase) cascade.

G proteins generally are signal transducing proteins, and they often have oncogen analogues. For example, the profoundly characterised oncogen ras codes for a protein that binds GTP normally but has no GTPase activity. If the corresponding Ras protein is formed in cells, it remains permanently (“constitutively”) activated, the signals of the normal receptors are ignored. This results in uncontrolled growth. Mutations in ras participate in 30 to 50% of all lung and colon carcinomas as well as more than 90% of the pancreas carcinomas.

Therefore, as GPP is used for the prenylation of proteins, blockers of FPPS will inhibit the activity of small GTPase oncoproteins involved in many cancers and modulate important pathways for regulating signal transduction. Additionally modulating this enzyme will have effects on cholesterol biosynthesis similar to those of the HMG CoA reductase inhibitors currently on the market.

FPPS was recently shown to be the molecular target of nitrogen-containing bisphosphonate drugs such as Aredia<< (pamidronate) and Zometa<< (zoledronic acid). Bisphosphonates are an established and very effective class of drugs that inhibit bone resorption by osteoclasts and are thus used for the treatment of conditions involving abnormally increased bone turnover, e.g. osteoporosis, Paget's disease, hypercalcemia and bone metastases. Hence, FPPS is now recognized as an important drug target. It is anticipated that new FPPS inhibitors would have therapeutic potential not only for the treatment of bone diseases but also in oncology, for the treatment of elevated cholesterol levels, and as anti-infectives.

In addressing the cellular mechanisms related to suppression of bone resorption, substantial evidence has accumulated to link loss of geranylgeranylation to induction of osteoclast apoptosis, disruption of the actin cytoskeleton and altered membrane trafficking (see e.g. F. P. Coxon et al., J. Bone Miner. Res. 2000, 15:1467). It was reported that bisphosphonates induce osteoclast apoptosis, both in vitro and in vivo, both in normal mice and in mice with increased bone resorption (see e.g. D. E. Huges et al., J. Bone Miner. Res. 1995, 10:1478). The apoptotic action of both nitrogen-containing bisphosphonates (N-BPs) and BPs lacking nitrogen results from intracellular action within the osteoclast, as opposed to other indirect actions via osteoblasts (see e.g. A. A. Reszka et al., JBC 1999, 274:34967). The likelihood that N-BPs cause apoptosis by interfering with isoprenylated proteins in osteoclasts was demonstrated by blocking the effect simply by replacing GGPP. Induction of osteoclast apoptosis by the N-BPs alendronate and risendronate, but not clodronate or etidronate, was blocked by addition of geranylgeraniol, but not farnesol, suggesting that only geranylgeranylation was critical. The signaling pathways involving geranylgeranylated small GTPases that are affected by bisphosphonates and that lead to osteoclast apoptosis remain to be determined. Prenylated small GTPases such as those of the Ras, Rho, Rac, Cdc42, and Rab families are important signaling proteins that regulate a variety of cell processes important for osteoclast function, including cytoskeletal arrangement, membrane ruffling, trafficking of intracellular vesicles, and apoptosis (Coleman M. L. et al. Cell Death Differ 2002, 9:493; Coxon F. P. et al. Calcif Tissue Int 2002, 72:80; Etienne-Manneville S. et el., Nature 2002, 420:629; Zerial M. et al., Nat Rev Mol Cell Biol, 2001, 2:107). Inhibition of the mevalonate pathway and loss of prenylated proteins could therefore account for most, if not all, of the various effects of N-BPs on osteoclasts that have been described. For example, loss of prenylation of Rho, Rac, or Cdc42 could lead to loss of the osteoclast ruffled border, which is absent in osteoclasts treated with bisphosphonates in vitro or in vivo (Sato M. et al. J Bone Miner Res 1990, 5:31). Because Rho, Rac, and Cdc42 are required for cytoskeletal organization in osteoclasts (Chellaiah M. A. et al., Biochim Biophys Acta 2000, 429:429), loss of prenylation of these small GTPases could also cause the loss of actin rings, a characteristic effect of bisphosphonate treatment (e.g. Sato M et al. J Clin Invest 1991, 88:2095). Loss of prenylation of Rab GTPases would cause disruption of vesicular trafficking in osteoclasts (Alakangas A. et al., Calcif Tissue Int 2002, 70:40), thereby affecting formation of the ruffled border, trafficking of lysosomal enzymes, and transcytosis of degraded bone matrix (Mulari M. T. et al., Traffic 2003 4:113; Nesbitt S. A. et al., Science 1997, 276:266; Salo J. et al., Science 1997, 276:270). Loss of prenylation of small GTPases such as Rac, and disruption of downstream signaling pathways promoting cell survival is also the likely route by which N-BPs induce osteoclast apoptosis (Glantschnig H. et al., Cell Death Differ 2003, 10:1165).

It is thus a goal of the present invention to provide novel FPPS inhibitors and methods of inhibition of FPPS-dependent disorders, in particular with advantageous pharmacological properties, such as enhanced efficacy, tolarability, oral bioavailability and/or pharmacokinetics.

General Description of the Invention

Surprisingly, it has now been found that salicylic acid derivatives can show FPPS inhibition although they are not bisphosphonates, and that they are appropriate for the treatment of diseases that depend on FPPS activity, especially against tumor and cancer diseases of soft and hard tissues, especially metastasis, e.g. bone metastasis, or as cholesterol-lowering agents. In addition, a large number of novel compounds of this class have been found that are FPPS inhibitors.

### DETAILED DESCRIPTION OF THE INVENTION

In a first aspect, the invention relates to a compound of the formula I,

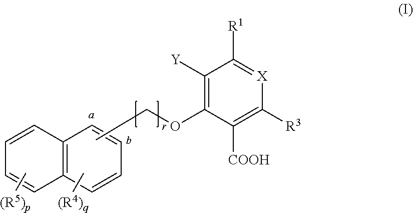

wherein R¹ is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group, with the proviso that not more than one of the pairs R¹ and R² or Y and R¹ form a bridge as defined above;
R³ is hydrogen, halo, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano;
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano;
p is an integer from 0, 1 to 4,
q is an integer from 0 to 3, and
r is 1 or 2,
or a pharmaceutically acceptable salt thereof,
for use in the treatment of a warm-blooded animal, especially a human, preferably for the treatment of an FPPS dependent disorder, the use of a compound of the formula I, or a pharmaceutically acceptable salt thereof, in the treatment of an FPPS dependent disease, the use of a compound of the formula I, or a pharmaceutically acceptable salt thereof, for the manufacture of a pharmaceutical preparation useful in the treatment of an FPPS dependent disease, a method of treatment comprising administering a compound of the formula I, or a pharmaceutically acceptable salt thereof, in a therapeutically effective amount to a warm-blooded animal, especially a human, especially where in need of such treatment, a pharmaceutical preparation for the treatment of an FPPS-dependent disease, comprising a compound of the formula I, or a pharmaceutically acceptable salt thereof, and a pharmaceutically acceptable carrier, and a method of preparing such a pharmaceutical preparation, comprising mixing a compound of the formula I, or a pharmaceutically acceptable salt thereof, with at least one pharmaceutically acceptable carrier material.

The general terms used hereinbefore and hereinafter preferably have, within this disclosure, the following meanings, unless otherwise indicated (where preferred embodiments can be defined by replacing one or more up to all general expressions or symbols with (a) more specific or more preferred definition(s) given herein):

Where the plural form is used for compounds, salts, pharmaceutical compositions, diseases and the like, this is intended to mean also a single compound, salt, or the like

The terms “treatment” or “therapy” refer to the prophylactic or preferably therapeutic (including but not limited to palliative, curing, symptom-alleviating, symptom-reducing, FPPS-activity-regulating and/or FPPS-inhibiting) treatment of said diseases/disorder, especially of the diseases/disorders mentioned below.

“A” compound, “a” salt, “a” disorder, “a” disease or the like preferably means “one or more” compounds, salt, disorders, diseases or the like.

“Obtainable by” can preferably be replaced with “obtained by”.

Where the term “comprising” is used, this is intended to mean that the component, components, action, actions, feature or features mentioned or enumerated thereafter may be fulfilled not only alone, but that also one or more other components and/or features (e.g. other additives, other actions) may be present in addition to those specifically mentioned. This is in contrast to the term “containing” or “consisting of” which here mean that no other components or features are included except for those specifically mentioned after such an expression and thus denote a complete enumeration/representation of features and/or components. Wherever “comprising” is used, this may (independently of other occurrences) be replaced by the narrower term “consisting of” or (in case of processes or methods) by “containing the step of”, where possible and expedient, thus leading to specific and preferred embodiments of the invention.

In unsubstituted or substituted alkyl, alkyl (also in alkoxy or the like) preferably has up to 20, more preferably up to 12 carbon atoms, is linear or branched, and is more preferably lower alkyl, especially C₁-C₄-alkyl. Substituted alkyl is preferably C₁- to C₂₀-alkyl, more preferably lower alkyl, that can be linear or branched one or more times (provided the number of carbon atoms allows this), e.g. methyl, ethyl, propyl, n-butyl, sec-butyl, isobutyl, tert-butyl, 2,2-dimethylpropyl, 1,2,2-trimethylpropyl, 1-ethyl-propyl, and that is substituted by one or more, preferably up to three, substitutents independently selected from the group consisting of unsubstituted or substituted heterocyclyl (preferably other than imidazol-1-yl) as described below, especially pyrrolidinyl, such as pyrrolidino, oxopyrrolidinyl, such as oxopyrrolidino, C₁-C₇-alkyl-pyrrolidinyl, 2,5-di-(C₁-C₇alkyl)pyrrolidinyl, such as 2,5-di-(C₁-C₇alkyl)pyrrolidino, tetrahydrofuranyl, thiophenyl, C₁-C₇-alkylpyrazolidinyl, pyridinyl, C₁-C₇-alkylpiperidinyl, piperidino, piperidino substituted by amino or N-mono- or N,N-di-[lower alkyl, phenyl, C₁-C₇-alkanoyl and/or phenyl-lower alkyl)-amino, unsubstituted or N-lower alkyl substituted piperidinyl bound via a ring carbon atom, piperazino, lower alkylpiperazino, morpholino, thiomorpholino, S-oxo-thiomorpholino or S,S-dioxothiomorpholino; unsubstituted or substituted aryl as defined below, especially phenyl, naphthyl, mono- to tri-[C₁-C₇-alkyl, halo and/or cyano]-phenyl or mono- to tri-[C₁-C₇-alkyl, halo and/or cyano]-naphthyl; unsubstituted or substituted cycloalkyl as defined below, especially C₃-C₈-cycloalkyl, mono- to tri-[C₁-C₇-alkyl and/or hydroxyl]-C₃-C₈-cycloalkyl; halo (e.g. in trifluoromethyl), hydroxy, lower alkoxy, lower-alkoxy-lower alkoxy, (lower-alkoxy)-lower alkoxy-lower alkoxy, halo-C₁-C₇-alkoxy, tri-(C₁-C₇-alkyl)silyl-C₁-C₇-alkoxy-C₁-C₇-alkoxy, phenoxy, naphthyloxy, phenyl- or naphthyl-lower alkoxy; amino-lower alkoxy, lower-alkanoyloxy, benzoyloxy, naphthoyloxy, nitro, cyano, formyl (CHO), carboxy, lower alkoxy carbonyl, e.g.; phenyl- or naphthyl-lower alkoxycarbonyl, such as benzyloxycarbonyl; C₁-C₇-alkanoyl, such as acetyl, benzoyl, naphthoyl, carbamoyl, N-mono- or N,N-disubstituted carbamoyl, such as N-mono- or N,N-di-substituted carbamoyl wherein the substitutents are selected from lower alkyl and hydroxy-lower alkyl; amidino, guanidino, ureido, mercapto, lower alkylthio, phenyl- or naphthylthio, phenyl- or naphthyl-lower alkylthio, lower alkyl-phenylthio, lower alkyl-naphthylthio, halogen-lower alkylmercapto, lower alkylsulfinyl, phenyl- or naphthyl-sulfinyl, phenyl- or naphthyl-lower alkylsulfinyl, lower alkyl-phenylsulfinyl, lower alkyl-napthyl-sulfinyl, sulfo, lower alkanesulfonyl, phenyl- or naphthyl-sulfonyl, phenyl- or naphthyl-lower alkylsulfonyl, alkylphenylsulfonyl, halogen-lower alkylsulfonyl, such as trifluoromethanesulfonyl; sulfonamido, benzosulfonamido, azido, azido-C₁-C₇-alkyl, especially azidomethyl, amino, amino-C₁-C₇-alkyl, especially aminomethyl, N-mono- or N,N-di-[lower alkyl, phenyl, C₁-C₇-alkanoyl and/or phenyl-lower alkyl)-amino or N-mono- or N,N-di-[lower alkyl, phenyl, C₁-C₇-alkanoyl and/or phenyl-lower alkyl)-aminomethyl; where each phenyl or naphthyl (also in phenoxy or naphthoxy) mentioned above as substituent or part of a substituent of substituted alkyl (or also of substituted aryl, heterocyclyl etc. mentioned herein) is itself unsubstituted or substituted by one or more, e.g. up to three, preferably 1 or 2, substituents independently selected from halo, especially fluoro, chloro, bromo or iodo, halo-lower alkyl, such as trifluoromethyl, hydroxy, lower alkoxy, azido, amino, N-mono- or N,N-di-(lower alkyl, phenyl, naphthyl, C₁-C₇-alkanoyl, phenyl-lower alkyl and/or naphthyl-lower alkyl)-amino, nitro, formyl (CHO), carboxy, lower-alkoxycarbonyl carbamoyl, cyano and/or sulfamoyl. In the case of R¹ in formula I, unsubstituted or substituted alkyl is preferably C₁-C₇-alkyl, such as methyl or ethyl, halo-C₁-C₇-alkyl, such as halomethyl, hydroxyl-C₁-C₇-alkyl, such as hydroxymethyl, amino-C₁-C₇-alkyl, such as aminomethyl, or carboxy-C₁-C₇-alkyl, such as carboxymethyl.

Unsubstituted or substituted alkenyl is preferably C₂-C₂₀-alkenyl, more preferably C₂-C₁₂-alkenyl, yet more preferably C₂-C₇-alkenyl, which is linear or branched and includes one or more double bonds. The substituents are preferably one or more, especially up to three, substituents independently selected from those mentioned for substituted alkyl, preferably with the proviso that substituents with active hydrogen (such as amino or hydroxyl) can also be present in tautomeric form (as keto or imino compounds) or are excluded from the substituents where the stability is too low.

Unsubstituted or substituted alkynyl is preferably C₂-C₂₀-alkynyl, more preferably C₃-C₁₂-alkynyl, yet more preferably C₃-C₇-alkynyl, which is linear or branched and includes one or more triple bonds. The substituents are preferably one or more, especially up to three, substituents independently selected from those mentioned for substituted alkyl, preferably with the proviso that substituents with active hydrogen (such as amino or hydroxyl) can also be present in tautomeric form (as keto or imino compounds) or are excluded from the substituents where the stability is too low.

In unsubstituted or substituted aryl, aryl is preferably an unsaturated carbocyclic system of not more than 20 carbon atoms, especially not more than 16 carbon atoms, is preferably mono-, bi- or tri-cyclic, e.g. phenyl, naphthyl, phenanthrenyl or fluorenyl, which is unsubstituted or, as substituted aryl, substituted preferably by one or more, preferably up to three, e.g. one or two substituents independently selected from those mentioned above for substituted alkyl, and from alkenyl. Preferably, the substituents are independently selected from the group consisting of C₁-C₇-alkyl, such as methyl, hydroxyl-C₁-C₇-alkyl, such as hydroxymethyl, halo, such as fluoro, chloro, bromo or iodo, hydroxyl, C₁-C₇-alkoxy, such as methoxy, halo-C₁-C₇-alkoxy, such as trifluoromethoxy, amino, C₁-C₇-alkanoylamino, such as acetylamino, amino-alkyl, such as aminomethyl, N-mono- or N,N-disubstituted amino-alkyl, preferably N-mono- or N,N-disubstituted amino-C₁-C₇-alkyl, such as N-mono- or N,N-disubstituted aminomethyl, and azidoalkyl, preferably azido-C₁-C₇-alkyl, such as azidomethyl, cyano or C₁-C₇-alkanoyl, especially CHO or from C₂-C₇-alkenyl.

In unsubstituted or substituted heterocyclyl, heterocyclyl is preferably a heterocyclic radical that is unsaturated (=carrying the highest possible number of conjugated double bonds in the ring(s)), saturated or partially saturated and is preferably a monocyclic or in a broader aspect of the invention bicyclic or tricyclic ring; and has 3 to 24, more preferably 4 to 16, most preferably 4 to 10 ring atoms; wherein one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; which heterocyclic radical (heterocyclyl) is unsubstituted or substituted by one or more, especially 1 to 3, substituents independently selected from the group consisting of the substituents defined above for substituted alkyl; and where heterocyclyl is especially a heterocyclyl radical selected from the group consisting of oxiranyl, azirinyl, aziridinyl, 1,2-oxathiolanyl, thienyl (=thiophenyl), furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidinyl, piperazinyl, pyridazinyl, morpholinyl, thiomorpholinyl, (S-oxo or S,S-dioxo)-thiomorpholinyl, indolizinyl, azepanyl, diazepanyl, especially 1,4-diazepanyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, benzo[1,3]dioxol-5-yl and 2,3-dihydro-benzo[1,4]dioxin-6-yl, each of these radicals being unsubstituted or substituted by one or more, preferably up to three, substitutents selected from those mentioned above for substituted alkyl, from alkenyl, e.g. C₁-C₇-alkenyl, and from oxo, especially from the group consisting of lower alkyl, especially methyl or tert-butyl, lower alkoxy, especially methoxy, oxo and halo.

In unsubstituted or substituted cycloalkyl, cycloalkyl is preferably a saturated mono- or bicyclic hydrocarbon group with 3 to 16, more preferably 3 to 9 ring carbon atoms, especially C₃-C₈-cycloalkyl, e.g. cyclopropyl, cyclobutyl, cyclopentyl, cyclohexyl, cycloheptyl or cyclooctyl, and is substituted by one or more, preferably one to three, substitutents independently selected from those described for substituted alkyl, especially from C₁-C₇-alkyl and hydroxy, or is (preferably) unsubstituted.

Halo(or halogen) is preferably fluoro, chloro, bromo or iodo, most preferably fluoro, chloro or bromo.

Carboxy is —COOH (here shown in the free form, may also form a salt).

In unsubstituted or substituted alkanoyl, alkanoyl is preferably formyl or more preferably C₂-C₂₀- yet more preferably C₂-C₇-alkanoyl, such as acetyl, propanoyl or butyroyl, is linear or branched and is substituted with one or more, especially up to three, substitutents independently selected from those mentioned above for substituted alkyl or is preferably unsubstituted as mentioned above, or is formyl (—CHO).

In unsubstituted or substituted aroyl, aroyl is preferably aryl-carbonyl (aryl-C(═O)—) wherein aryl is defined as above, e.g. benzoyl or naphthoyl, and is unsubstituted or substituted by one or more, preferably up to three, substituents independently selected from those mentioned above for alkyl.

In unsubstituted or substituted heterocyclylcarbonyl (heterocyclyl-C(═O)—), heterocyclyl is preferably as defined above an is unsubstituted or preferably substituted by one or more, especially up to three, moieties independently selected from those mentioned above for substituted alkyl and from oxo.

In amino-alkyl (also a special variant of substituted alkyl), alkyl is preferably as defined above and is unbranched or branched. The amino moiety is preferably bound to a terminal carbon atom. Preferred is amino-C₁-C₇-alkyl, especially aminomethyl.

In N-mono- or N,N-disubstituted amino-alkyl, alkyl is preferably as defined above and is unbranched or branched. The mono- or disubstituted amino moiety is preferably bound to a terminal carbon atom. The substituents are preferably selected from unsubstituted or substituted alkyl, especially C₁-C₇-alkyl or phenyl-C₁-C₇-alkyl, such as methyl, ethyl or benzyl, acyl, especially C₁-C₇-alkanoyl, such as acetyl, unsubstituted or substituted aryl, preferably as defined above, especially phenyl, unsubstituted or substituted aroyl, preferably as defined above, e.g. benzoyl, and unsubstituted or substituted cycloalkyl, preferably as defined above, especially cyclopropyl, cyclobutyl, cyclopentyl or cyclohexyl.

In azido-alkyl (also a special variant of substituted alkyl), alkyl is preferably as defined above and is unbranched or branched. The azido moiety is preferably bound to a terminal carbon atom. Preferred is azido-C₁-C₇-alkyl, especially azidomethyl.

That R¹ and R² together form a bridge of the formula #CH═CH—CH═C#H bound at the carbons marked with #, thus forming an annealed benzo group, means that together with this benzo group the ring binding R¹ and R² represents a naphthyl moiety.

That Y together with R¹ forms a bridge of the formula #CH═CH—CH═C#H bound at the carbons marked with #, thus forming an annealed benzo group, means that together with this benzo group the ring binding Y and R¹ represents a naphthyl moiety.

Etherified hydroxyl is preferably unsubstituted or substituted (preferably C₁-C₇-) alkyloxy, wherein the substituents are preferably independently selected from those mentioned for substituted alkyl, preferably methoxy or 3-(2-trimethylsilyl-ethoxy-methoxy; or is unsubstituted or substituted aryloxy wherein unsubstituted or substituted aryl is as defined above; e.g. substituted or preferably unsubstituted phenyloxy or naphthyloxy, respectively.

Esterified hydroxyl is preferably acyloxy with acyl as defined below, more preferably C₁-C₇-alkanoyloxy, such as acetoxy, benzoyloxy, naphthoyloxy, C₁-C₇-alkansulfonyloxy (alkyl-S(O)₂—O—), or phenyl- or naphthylsulfonyloxy (phenyl-S(O)₂—O— or naphthyl-S(O)₂—O—) wherein phenyl is unsubstituted or substituted, e.g. by one or more, e.g. up to 3, C₁-C₇-alkyl moieties.

Acyl is preferably unsubstituted or substituted aryl-carbonyl (=aryl-CO—; =aroyl) or -sulfonyl (=aryl-S(O)₂—), unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, unsubstituted or substituted alkyloxycarbonyl or -oxysulfonyl, unsubstituted or substituted aryl-oxycarbonyl or -oxysulfonyl, unsubstituted or substituted heterocyclyloxycarbonyl or -oxysulfonyl, or unsubstituted or substituted cycloalkyloxycarbonyl or -oxysulfonyl wherein unsubstituted or substituted aryl, unsubstituted or substituted heterocyclyl, unsubstituted or substituted cycloalkyl and unsubstituted or substituted alkyl are preferably as described above. Preferred is C₁-C₇-alkanoyl, such as acetyl, unsubstituted or mono-, di- or tri-(halo and/or C₁-C₇-alkyl)-substituted benzoyl or naphthoyl, C₃-C₈-cycloalkylcarbonyl, pyrrolidincarbonyl, especially pyrrolidinocarbonyl, C₁-C₇-alkylsulfonyl, such as methylsulfonyl (=methanesulfonyl), (phenyl- or naphthyl)-C₁-C₇-alkylsulfonyl, such as phenylmethansulfonyl, or (unsubstituted, or [C₁-C₇-alkyl-, phenyl-, halo-lower alkyl-, halo, oxo-C₁-C₇-alkyl- C₁-C₇-alkyloxy-, phenyl-C₁-C₇-alkoxy-, halo-C₁-C₇-alkyloxy-, phenoxy-, C₁-C₇-alkanoylamino-, cyano-, C₁-C₇-alkanoyl- and/or C₁-C₇-alkylsulfonyl-]substituted) (phenyl or naphthyl)-sulfonyl, such as phenylsulfonyl (=benzenesulfonyl), naphthalene-1-sulfonyl, naphthalene-2-sulfonyl or toluene-4-sulfonyl, or (C₁-C₇-alkyl, phenyl, naphthyl, phenyl-C₁-C₇-alkyl and/or napthyl-C₁-C₇-alkyl)-oxycarbonyl, e.g. C₁-C₇-alkoxycarbonyl, such as methoxycarbonyl.

Of the symbols indicating integers, p is an integer from 0 to 4, preferably 0, 1 or 2; q is an integer from 0 to 3, preferably 0, 1 or 2 and r is 1 or 2, preferably 1.

In some cases, a compound of the present invention may comprise one or more chiral centers in substitutents or show other asymmetry (leading to enantiomers) or may otherwise be able to exist in the form of more than one stereoisomer, e.g. due more than one chiral centers or more than one other type of asymmetry or due to rings or double bonds that allow for Z/E (or cis-trans) isomerism (diastereomers). The present inventions includes both mixtures of two or more such isomers, such as mixtures of enantiomers, especially racemates, as well as preferably purified isomers, especially purified and most especially essentially (that is at least more than 90%) pure enantiomers or diastereomers, or enantiomerically enriched mixtures.

If in formula I the napthyl moiety is bound to the rest of the molecule via its carbon marked with “b” in formula I, the result is a compound of the formula IA

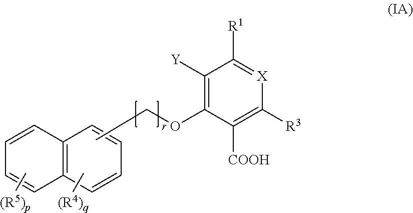

which represent one embodiment of a compound of the formula I.

If in formula I the napthyl moiety is bound to the rest of the molecule via its carbon marked with “a” in formula I, the result is a compound of the formula IB

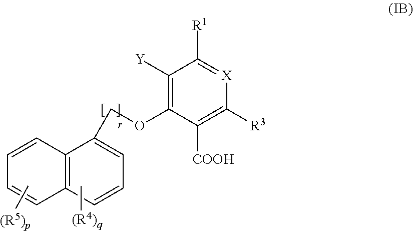

which represents a preferred embodiment of a compound of the formula I of the various embodiments according to the invention.

### PREFERRED EMBODIMENTS OF THE INVENTION

In the following preferred embodiments of the moieties and symbols in formula I, the more specific definitions given above can be employed independently of each other to replace more general definitions and thus to define specially preferred embodiments of the invention, where the remaining definitions can be kept broad as defined in embodiments of the invention defined above of below.

R¹ is preferably hydrogen or (especially in novel compounds of the formula I) C₁-C₇-alkyl, amino-C₁-C₇-alkyl, N-mono- or N,N-di-(C₁-C₇-alkyl, phenyl, C₁-C₇-alkanoyl and/or phenyl-lower alkyl)-amino-C₁-C₇-alkyl, halo-C₁-C₇-alkyl, halo, C₁-C₇-alkoxy, hydroxy-C₁-C₇-alkoxy, carboxy-C₁-C₇-alkoxy, halo-C₁-C₇-alkoxy, phenyl- or naphthyl-C₁-C₇-alkoxy, amino, N-mono- or N,N-di-{C₁-C₇-alkyl, hydroxy-C₁-C₇-alkyl, C₁-C₇-alkoxy-C₁-C₇-alkyl, [N′,N′-di-(C₁-C₇-alkyl)amino-C₁-C₇-alkyl, C₃-C₈-cycloalkyl, mono- to tri-[C₁-C₇-alkyl and/or hydroxyl-C₃-C₈-cycloalkyl, phenyl, naphthyl, mono- to tri-[C₁-C₇-alkyl, halo and/or cyano]-phenyl, mono- to tri-[C₁-C₇-alkyl, halo and/or cyano]-naphthyl, C₁-C₇-alkanoyl, phenyl-C₁-C₇-alkyl, phenyl-C₁-C₇-alkyl, C₃-C₈-cycloalkyl-C₁-C₇-alkyl, [(C₁-C₇-alkyl)-C₃-C₈-cycloalkyl]-C₁-C₇-alkyl, [hydroxy-C₃-C₈-cycloalkyl]-C₁-C₇-alkyl, [C₁-C₇-alkyl-pyrrolidinyl]-C₁-C₇-alkyl, [tetrahydrofuranyl-C₁-C₇-alkyl, thiophenyl-C₁-C₇-alkyl, pyridinyl-C₁-C₇-alkyl, C₁-C₇-alkylpyrazolidinyl, pyridinyl and/or C₁-C₇-alkylpiperidinyl}-amino, (or especially) phenyl, naphthyl, mono-, di- or tri-(C₁-C₇-alkyl)phenyl or -naphthyl, hydroxyl-C₁-C₇-alkyl-phenyl or -naphthyl, mono-, di- or tri-(halo)-phenyl or -naphthyl, hydroxyphenyl, hydroxynaphthyl, mono-, di- or tri-(C₁-C₇-alkoxy)-phenyl or naphthyl, halo-C₁-C₇-alkoxy-phenyl or -naphthyl (e.g. trifluoromethoxyphenyl), C₁-C₇-alkanoyl-phenyl or -naphthyl, azido-C₁-C₇-alkylphenyl, amino-C₁-C₇-alkylphenyl, benzo[1,3]dioxolyl, 2,3-dihydro-benzo[1,4]dioxinyl, pyrrolyl, 2,5-di-(C₁-C₇-alkyl)-pyrrolyl, pyrrolidinyl, oxopyrrolidinyl, mono- or di-C₁-C₇-alkylpyrrolidinyl, furanyl, piperidinyl, C₁-C₇-alkoxypyridinyl, hydroxy-C₁-C₇-alkylpiperidinyl (especially -piperidino), piperazinyl, especially piperazino, C₁-C₇-alkylpiperazinyl, especially -piperazino, C₁-C₇-alkyl-piperazinyl, especially -piperazinyl, morpholinyl, especially morpholino, thiomorpholinyl, especially thiomorpholino, S-oxo-thiomorpholinyl, especially S-oxo-thiomorpholino, S,S-dioxothiomorpholinyl, especially S,S-dioxo-thiomorpholino, azepanyl, especially azepan-1-yl, C₁-C₇-alkyl-1,4-diazepanyl, especially -diazepan-1-yl, indolyl, indolyl, N-(C₁-C₇-alkyl)indolyl, benzofuranyl or benzothiophenyl.
X is preferably CR² wherein R² is hydrogen or, if the napthyl ring is bound to the rest of the molecule in formula I via its carbon marked “a”, hydrogen or halo, preferably hydrogen;
or X is CR² and R¹ and R² together form a bridge of the formula #CH═CH—CH═C#H bound at the carbons marked with #, thus completing an annealed benzo group.
Y is preferably hydrogen or together with R¹ forms a bridge of the formula #CH═CH—CH═C#H bound at the carbons marked with #, thus completing an annealed benzo group.
R³ is preferably hydrogen or hydroxyl.

Each R⁴ and/or R⁵ if present, in the case that more than one moiety R⁴ and/or R⁵ is present independently of the others, is C₁-C₇-alkyl, hydroxy-C₁-C₇-alkyl, hydroxy, tri-(C₁-C₇-alkylsilyl)-C₁-C₇-alkoxy-C₁-C₇-alkoxy, halo, C₁-C₇-alkoxycarbonyl or cyano.

The index number p and/or (if present which is only possible if n is 1) the index number q is preferably 0, 1 or 2, respectively, more preferably with the proviso that the sum of p and q is 0, 1, or 2.

The index number r is 2 or preferably 1.

In one type of the embodiments of the invention, if R¹ is substituted alkyl or H, then the sum of p and q is 1 or larger (p+q≧1).

In one type of the embodiments of the invention, R¹ and Y have a meaning defined above or below other than a bridge of the formula #CH═CH—CH═C#H.

In one type of the embodiments of the invention, at least one of Y, R¹ and R³ is other than hydrogen and either r is 1 or R¹ is other than substituted alkyl (meaning it has a meaning mentioned herein for R¹ different from substituted alkyl), or r is 1 and R¹ is other than substituted alkyl.

The invention also relates to a novel compound of the formula I as defined above or below, as such, or a salt thereof, with the proviso that the compounds are other than 4-(imidazol-1-ylmethyl)-2-[2-(napthalin-1-yl)ethoxy]-benzoic acid, 3-(napthalin-2-ylmethoxy)-2-naphthoic acid and 2-(naphthalin-1-ylmethoxy)-benzoic acid.

In one important embodiment, the invention relates to a compound of the formula IB shown above wherein

R¹ is hydrogen, unsubstituted alkyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted cycloalkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or halo,
or X is CR² and R¹ and R² together form a bridge of the formula #CH═CH—CH═C#H bound at the carbons marked with #, thus completing an annealed benzo group,
with R² preferably being hydrogen;
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
p is an integer from 0 to 4,
q is an integer from 0 to 3, and
r is 1 or 2,
with the proviso that at least one of R¹, R² and R³ must have a meaning mentioned for the present embodiment other than hydrogen;
or a pharmaceutically acceptable salt thereof.

Especially preferred among the compounds mentioned in the preceding paragraph is a compound of the formula IB, wherein at least one of p and q is one, or a pharmaceutically acceptable salt thereof.

In another important embodiment, the invention relates to novel compounds of the formula I, especially IA or more especially IB, shown above wherein

R¹ is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted cycloalkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
p is an integer from 0 to 4,
q is an integer from 0 to 3, and
r is 1 or 2,
with the proviso (i) that if R¹ is hydrogen, X is C—R² wherein R² is hydrogen, R³ is hydrogen, n is 1, q is 0 and p is 1, then R⁵ is substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, carboxy, acyl, nitro or cyano (that is, not unsubstituted alkyl); and with the proviso (ii) that if R¹ has one of the meanings defined above for this embodiment/claim other than hydrogen, and X, Y, R², R³, R⁴, n, q and r are as defined in this embodiment/claim before the provisos (i) and (ii), then p can also be 0 (zero) (that is only hydrogen is present, not a substituent R⁵) or p can also be 1 and R⁵ can also be unsubstituted alkyl;
or a pharmaceutically acceptable salt thereof;
or especially their use according to the invention.

Another important embodiment relates to a novel compound of the formula I, especially IA or more especially Formula IB shown above wherein

R¹ is unsubstituted alkyl; substituted alkyl selected from the group consisting of amino-C₁-C₇-alkyl, N-mono- or N,N-di-(C₁-C₇-alkyl, phenyl, C₁-C₇-alkanoyl and/or phenyl-lower alkyl)amino-C₁-C₇-alkyl and halo-C₁-C₇-alkyl; unsubstituted or substituted alkenyl, unsubstituted or substituted alkynyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted cycloalkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino,
p is an integer from 0 to 4,
q is an integer from 0 to 3, and
r is 1 or 2,
or a pharmaceutically acceptable salt thereof;
or especially the use according to the invention.

A still more preferred embodiment relates to a novel compound of the formula I, especially formula IA or more especially formula IB, shown above wherein

R¹ is unsubstituted alkyl; substituted alkyl selected from the group consisting of amino-C₁-C₇-alkyl, N-mono- or N,N-di-(C₁-C₇-alkyl, phenyl, C₁-C₇-alkanoyl and/or phenyl-lower alkyl)amino-C₁-C₇-alkyl and halo-C₁-C₇-alkyl; unsubstituted or substituted aryl, unsubstituted or substituted heterocyclyl, halo, —OR or —NR₂ wherein
R is, independently of one another if present twice, hydrogen, unsubstituted or substituted alkyl, unsubstituted or substituted cycloalkyl, unsubstituted or substituted aryl or unsubstituted or substituted heterocyclyl,
X is CR² wherein R² is hydrogen, if the napthyl ring is bound to the rest of the molecule in formula I via its carbon marked “a”, hydrogen or or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl;
each R⁴ if present, in the case that more than one moiety R⁴ is present independently of the others, is unsubstituted or substituted alkyl, hydroxyl, etherified hydroxyl, halo or cyano,
each R⁵ if present, in the case that more than one moiety R⁵ is present independently of the others, is unsubstituted or substituted alkyl, hydroxyl, etherified hydroxyl, halo or cyano,
p is an integer from 0 to 4,
q is an integer from 0 to 3, and
r is 1 or 2,
or a pharmaceutically acceptable salt thereof; or especially the use according to the invention.

In another preferred embodiment, the invention relates to a compound of the formula I, especially the formula IB, wherein

R¹ is hydrogen, C₁-C₇-alkyl, amino-C₁-C₇-alkyl, N-mono- or N,N-di-(C₁-C₇-alkyl, phenyl, C₁-C₇-alkanoyl and/or phenyl-lower alkyl)-amino-C₁-C₇-alkyl, halo-C₁-C₇-alkyl (e.g. trifluoromethyl), halo, C₁-C₇-alkoxy, hydroxy-C₁-C₇-alkoxy, carboxy-C₁-C₇-alkoxy, halo-C₁-C₇-alkoxy, phenyl- or naphthyl-C₁-C₇-alkoxy, amino, N-mono- or N,N-di-{C₁-C₇-alkyl, hydroxy-C₁-C₇-alkyl, C₁-C₇-alkoxy-C₁-C₇-alkyl, [N′,N′-di-(C₁-C₇-alkyl)amino-C₁-C₇-alkyl, C₃-C₈-cycloalkyl, mono- to tri-[C₁-C₇-alkyl and/or hydroxy]-C₃-C₈-cycloalkyl, phenyl, naphthyl, mono- to tri-[C₁-C₇-alkyl, halo and/or cyano]-phenyl, mono- to tri-[C₁-C₇-alkyl, halo and/or cyano]-naphthyl, C₁-C₇-alkanoyl, phenyl-C₁-C₇-alkyl, phenyl-C₁-C₇-alkyl, C₃-C₈-cycloalkyl-C₁-C₇-alkyl, [(C₁-C₇-alkyl)-C₃-C₈-cycloalkyl]-C₁-C₇-alkyl, [hydroxy-C₃-C₈-cycloalkyl]-C₁-C₇-alkyl, [C₁-C₇-alkyl-pyrrolidinyl]-C₁-C₇-alkyl, [tetrahydrofuranyl-C₁-C₇-alkyl, thiophenyl-C₁-C₇-alkyl, pyridinyl-C₁-C₇-alkyl, C₁-C₇-alkylpyrazolidinyl, pyridinyl and/or C₁-C₇-alkylpiperidinyl]-amino, (or especially) phenyl, naphthyl, mono-, di- or tri-(C₁-C₇-alkyl)-phenyl or -naphthyl, hydroxyl-C₁-C₇-alkyl-phenyl or -naphthyl, mono-, di- or tri-(halo)-phenyl or -naphthyl, hydroxyphenyl, hydroxynaphthyl, mono-, di- or tri-(C₁-C₇-alkoxy)-phenyl or -naphthyl, halo-C₁-C₇-alkoxyphenyl or -naphthyl (e.g. trifluoromethoxyphenyl), C₁-C₇-alkanoyl-phenyl or -naphthyl, azido-C₁-C₇-alkylphenyl, amino-C₁-C₇-alkylphenyl, benzo[1,3]dioxolyl, 2,3-dihydro-benzo[1,4]dioxinyl, pyrrolyl, 2,5-di-(C₁-C₇-alkyl)pyrrolyl, pyrrolidinyl, oxopyrrolidinyl, mono- or di-C₁-C₇-alkylpyrrolidinyl, furanyl, piperidinyl, C₁-C₇-alkoxypyridinyl, hydroxy-C₁-C₇-alkylpiperidinyl (especially -piperidino), piperazinyl, especially piperazino, C₁-C₇-alkylpiperazinyl, especially -piperazino, C₁-C₇-alkyl-piperazinyl, especially -piperazinyl, morpholinyl, especially morpholino, thiomorpholinyl, especially thiomorpholino, S-oxo-thiomorpholinyl, especially S-oxo-thiomorpholino, S,S-dioxothiomorpholinyl, especially S,S-dioxo-thiomorpholino, azepanyl, especially azepan-1-yl, C₁-C₇-alkyl-1,4-diazepanyl, especially -diazepan-1-yl, indolyl, indolyl, N-(C₁-C₇-alkyl)-indolyl, benzofuranyl or benzothiophenyl,
X is CR² wherein R² is hydrogen or, if the napthyl ring is bound to the rest of the molecule in formula I via its carbon marked “a”, hydrogen or halo, preferably hydrogen;
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl,
each R⁴ and/or R⁵ if present, in the case that more than one moiety R⁴ and/or R⁵ is present independently of the others, is C₁-C₇-alkyl, hydroxy-C₁-C₇-alkyl, hydroxy, tri-(C₁-C₇-alkylsilyl)-C₁-C₇-alkoxy-C₁-C₇-alkoxy, halo, C₁-C₇-alkoxycarbonyl or cyano;
p is 0, 1 or 2,
q is 0, 1 or 2 and
r is 1 or 2, preferably 1,
or a pharmaceutically acceptable salt thereof;
as such (then with the proviso that R¹ has a meaning given in this embodiment other than hydrogen) or (without this latter proviso) for use in the treatment of a warm-blooded animal, especially a human, preferably for the treatment of an FPPS dependent disorder, the use of a compound of the formula I, or a pharmaceutically acceptable salt thereof, in the treatment of an FPPS dependent disease, the use of a compound of the formula I, or a pharmaceutically acceptable salt thereof, for the manufacture of a pharmaceutical preparation useful in the treatment of an FPPS dependent disease, a method of treatment comprising administering a compound of the formula I, or a pharmaceutically acceptable salt thereof, in a therapeutically effective amount to a warm-blooded animal, especially a human, especially where in need of such treatment, a pharmaceutical preparation for the treatment of an FPPS-dependent disease, comprising a compound of the formula I, or a pharmaceutically acceptable salt thereof, and a pharmaceutically acceptable carrier, and a method of preparing such a pharmaceutical preparation, comprising mixing a compound of the formula I, or a pharmaceutically acceptable salt thereof, with at least one pharmaceutically acceptable carrier material.

Yet more preferably, the invention relates to a compound of the formula I, especially Formula IB, wherein

R¹ is C₁-C₇-alkyl, amino-C₁-C₇-alkyl, N-mono- or N,N-di-(C₁-C₇-alkyl, phenyl, C₁-C₇-alkanoyl and/or phenyl-lower alkyl)-amino-C₁-C₇-alkyl, halo-C₁-C₇-alkyl, halo, C₁-C₇-alkoxy, hydroxy-C₁-C₇-alkoxy, carboxy-C₁-C₇-alkoxy, halo-C₁-C₇-alkoxy, phenyl- or naphthyl-C₁-C₇-alkoxy, amino, N-mono- or N,N-di-{C₁-C₇-alkyl, hydroxy-C₁-C₇-alkyl, C₁-C₇-alkoxy-C₁-C₇-alkyl, [N′,N′-di-(C₁-C₇-alkyl)-amino-C₁-C₇-alkyl, C₃-C₈-cycloalkyl, mono- to tri-[C₁-C₇-alkyl and/or hydroxy]-C₃-C₈-cycloalkyl, phenyl, naphthyl, mono- to tri-[C₁-C₇-alkyl, halo and/or cyano]-phenyl, mono- to tri-[C₁-C₇-alkyl, halo and/or cyano]-naphthyl, C₁-C₇-alkanoyl, phenyl-C₁-C₇-alkyl, phenyl-C₁-C₇-alkyl, C₃-C₈-cycloalkyl-C₁-C₇-alkyl, [(C₁-C₇-alkyl)-C₃-C₈-cycloalkyl]-C₁-C₇-alkyl, [hydroxy-C₃-C₈-cycloalkyl]-C₁-C₇-alkyl, [C₁-C₇-alkyl-pyrrolidinyl]-C₁-C₇-alkyl, [tetrahydrofuranyl-C₁-C₇-alkyl, thiophenyl-C₁-C₇-alkyl, pyridinyl-C₁-C₇-alkyl, C₁-C₇-alkylpyrazolidinyl, pyridinyl and/or C₁-C₇-alkylpiperidinyl]-amino, (or especially) phenyl, naphthyl, mono-, di- or tri-(C₁-C₇-alkyl)-phenyl or -naphthyl, hydroxyl-C₁-C₇-alkyl-phenyl or -naphthyl, mono-, di- or tri-(halo)-phenyl or -naphthyl, hydroxyphenyl, hydroxynaphthyl, mono-, di- or tri-(C₁-C₇-alkoxy)-phenyl or -naphthyl, halo-C₁-C₇-alkoxy-phenyl or -naphthyl (e.g. trifluoromethoxyphenyl), C₁-C₇-alkanoyl-phenyl or -naphthyl, azido-C₁-C₇-alkylphenyl, amino-C₁-C₇-alkylphenyl, benzo[1,3]dioxolyl, 2,3-dihydro-benzo[1,4]dioxinyl, pyrrolyl, 2,5-di(C₁-C₇-alkyl)pyrrolyl, pyrrolidinyl, oxopyrrolidinyl, mono- or di-C₁-C₇-alkylpyrrolidinyl, furanyl, piperidinyl, C₁-C₇-alkoxypyridinyl, hydroxy-C₁-C₇-alkylpiperidinyl (especially -piperidino), piperazinyl, especially piperazino, C₁-C₇-alkylpiperazinyl, especially -piperazino, C₁-C₇-alkylpiperazinyl, especially -piperazinyl, morpholinyl, especially morpholino, thiomorpholinyl, especially thiomorpholino, S-oxo-thiomorpholinyl, especially S-oxo-thiomorpholino, S,S-dioxothiomorpholinyl, especially S,S-dioxo-thiomorpholino, azepanyl, especially azepan-1-yl, C₁-C₇-alkyl-1,4-diazepanyl, especially -diazepan-1-yl, indolyl, indolyl, N-(C₁-C₇-alkyl)indolyl, benzofuranyl or benzothiophenyl,
X is CR² wherein R² is hydrogen, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl,
each R⁴ and/or R⁵ if present, in the case that more than one moiety R⁴ and/or R⁵ is present independently of the others, is C₁-C₇-alkyl, hydroxy-C₁-C₇-alkyl, hydroxy, halo, C₁-C₇-alkoxycarbonyl, cyano or tri-(C₁-C₇-alkylsilyl)-C₁-C₇-alkoxy-C₁-C₇-alkoxy;
p is 0, 1 or 2,
q is 0, 1 or 2 and
r is 1 or 2, preferably 1,
or a pharmaceutically acceptable salt thereof.

The invention especially relates to a compound of the formula I, especially of the formula IB, wherein

R¹ is methyl, aminomethyl, trifluoromethyl, phenyl, 2-methylphenyl, 3-methylphenyl, 2,4-dimethyl-phenyl, 2,6-dimethylphenyl, 3-(hydroxymethyl)phenyl, 4-(hydroxymethyl)-phenyl, 2-fluorophenyl, 3-fluorophenyl, 4-fluorophenyl, 3,4-difluoro-phenyl, 4-chlorophenyl, 3,4-dichlorophenyl, 3-hydroxyphenyl, 4-hydroxyphenyl, 2-methoxyphenyl, 3-methoxyphenyl, 2,6-dimethoxy-phenyl, 3,4-dimethoxyphenyl, 3-trifluoromethoxyphenyl, 4-acetylaminophenyl, 3-formylphenyl, 3-azidomethylphenyl, 3-aminomethylphenyl, benzo[1,3]dioxol-5-yl, 2,3-dihydro-benzo[1,4]dioxin-6-yl, pyrrol-1-yl, 2,5-dimethyl-pyrrol-1-yl, pyrrolidino, 2-oxopyrrolidino, 2,5-dimethylpyrrolidino, furan-3-yl, piperidino, 3-hydroxymethylpiperidino, piperazino, 4-methylpiperazino, 4-acetyl-piperazino, morpholino, 2-methoxy-pyridin-3-yl, azepan-1-yl, 4-methyl-1,4-diazepan-1-yl, indol-5-yl, indol-4-yl, N-methyl-indol-5-yl, 1-benzofuran-2-yl, 1-benzothiophen-3-yl, bromo, chloro, methoxy, 3-methyl-n-butoxy, 2-hydroxy-ethoxy, carboxymethyloxy, trifluoromethoxy, benzyloxy, N-methylamino, N-ethylamino, N,N-dimethylamino, N,N-diethylamino, N-(n-propyl)-amino, N-(2,2-dimethylpropyl)-amino, N-(1,2,2-trimethylpropyl)-amino, N-(1-(ethyl)-n-propyl)-amino, N-(2-hydroxyethyl)-amino, N-(3-hydroxypropyl)-amino, N-(2-methoxyethyl)-amino, N-(3-methoxypropyl)-amino, N-(2-methoxy-1-methyl-ethyl)amino*, N-(2-methoxyethyl)-N-methylamino, N-(2-hydroxyethyl)-N-methyl-amino, N-benzylamino, N-cyclopropylmethyl-amino, N-cyclohexylmethyl-amino, N-(1-cyclohexyl-ethan-1-yl)-amino*, N-cyclopropylmethyl-N-(n-propyl)-amino, N-[2-(N′,N′-diethylamino)-ethyl]-N-methyl-amino, N-phenylamino, N-(2-methylphenyl-amino, N-(4-methylphenyl)-amino, N-(2,6-dimethylphenyl)-amino, N-(naphthalin-2-yl)-amino, N-(4-isopropylphenyl)-amino, N-(2-fluorophenyl)-amino, N-(2-chlorophenyl)-amino, N-(3-chlorophenyl)-amino, N-(2-cyanophenyl)-amino, N-(3-chlorphenyl)-N-methyl-amino, N-(cyclobutyl)-amino, N-(cyclopentyl)-amino, N-(cycloheptyl)amino, N-(4-methyl-cyclohexyl)-amino*, N-(4-hydroxycyclohexyl)-amino,* N-[3-(1-methylpyrrolidin-2-yl)-propyl]-amino, N-(tetrahydrofuran-2-ylmethyl)-amino, N-(thiophen-2-ylmethyl)-amino, N-[2-(pyridin-2-yl)-ethyl]-N-methyl-amino, N-(1-methylpyrazolidin-5-yl)amino, N-(pyridin-2-yl)-amino, N-(pyridin-3-yl)-amino, N-(pyridin-4-yl)amino or N-(1-methylpiperidin-4-yl)-amino (where the moieties with an asterisk (*) can preferably be present in a form where each chiral carbon is present in isomerically pure form, that is, as R- or S-form);
X is CR² wherein R² is hydrogen or, if the napthyl ring is bound to the rest of the molecule in formula I via its carbon marked “a”, hydrogen, chloro or bromo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl,
each R⁴ and/or R⁵ if present, in the case that more than one moiety R⁴ and/or R⁵ is present independently of the others, is methyl, hydroxymethyl, hydroxyl, 3-(2-trimethylsilyl-ethoxymethoxy, chloro, methoxycarbonyl or cyano;
p is 0 or 1,
q is 0 or 1 and
r is 1,
or a pharmaceutically acceptable salt thereof.

In a highly preferred embodiment, the invention also relates to a novel compound of the formula I, or a (preferably pharmaceutically acceptable) salt thereof, as described in the Examples.

Other preferred embodiments are mentioned above and below or in the claims which are incorporated by reference herein.

### Process of Manufacture

A compound of the formula I can be obtained according to procedures that, in principle, are known in the art for analogous products, which for the novel compounds of the formula I are novel processes, especially by

hydrolyzing a compound of the formula II,

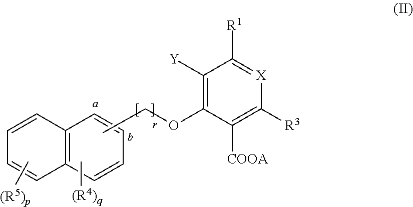

wherein R¹, R², R³, R⁴, R⁵, X, Y, n, p, q and r are as defined for a compound of the formula I and A is unsubstituted or substituted alkyl, preferably lower alkyl;
where in the starting material of the formula II additional functional groups present that shall not participate in this or a preceding reaction can be present in protected form and in the obtainable compounds of the formula I carrying one or more protecting groups such protecting groups are removed;
and, if desired, converting an obtainable compound of the formula I into a different compound of the formula I, converting an obtainable salt of a compound of the formula I into a different salt thereof, converting an obtainable free compound of the formula I into a salt thereof, and/or separating an obtainable isomer of a compound of the formula I from one or more different obtainable isomers of the formula I.

The hydrolysis can take place in the presence of an acid, especially of a hydrohalic acid, such as hydrochloric acid, in an appropriate solvent or mixture of solvents, e.g. in dioxane, e.g. at a temperature in the range from 0° C. to the boiling temperature of the reaction mixture, e.g. from 10° C. to 80° C.; or of a base, especially an alkalimetal hydroxide, such as lithium hydroxide, in an appropriate solvent or solvent mixture, such as tetrahydrofuran, water and/or methanol, preferably at temperatures in the range from 0° C. to the boiling temperature of the reaction mixture, e.g. from 10 to 80° C.

### Protecting Groups

If one or more other functional groups, for example carboxy, hydroxy, amino or the like are or need to be protected in a starting material of the formula II or any precursor, because they should not take part in the reaction or disturb the reaction, these are such groups as are usually used in the synthesis of peptide compounds, and also of cephalosporins and penicillins, as well as nucleic acid derivatives and sugars. Protecting groups are such groups that are no longer present in the final compounds once they are removed, while groups that remain as substitutents are not protecting groups in the sense used here which is groups that are added at a starting material or intermediate stage and removed to obtain a final compound. For example, tert-butoxy if remaining in a compound of the formula I is a substituent, while if it is removed to obtain the final compound of the formula I it is a protecting group.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by acetolysis, protonolysis, solvolysis, reduction, photolysis or also by enzyme activity, for example under conditions analogous to physiological conditions, and that they are not present in the end-products. The specialist knows, or can easily establish, which protecting groups are suitable with the reactions mentioned above and below.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

### Optional Reactions and Conversions

A compound of the formula I may be converted into a different compound of the formula I.

For example, in a compound of the formula I wherein a substituent R⁴ and/or R⁵ is present which is carboxy, said carboxy can be reduced to hydroxymethyl, e.g. by treatment first with ethylchloroformate in the presence of a tertiary nitrogen base, such as triethylamine or diisopropylethylamine, in an appropriate solvent, e.g. a cyclic ether, such as tetrahydrofuran, preferably at temperatures in the range from −50° C. to 30° C., followed by treatment with a reducing agent, e.g. sodium borohydride, in an appropriate solvent or solvent mixture, such as an alcohol, e.g. methanol, preferably at a temperature in the range from −50 to 20° C., e.g. from −20 to 10° C.

Also in the optional process steps, carried out “if desired”, functional groups of the starting compounds which should not take part in the reaction may be present in unprotected form or may be protected for example by one or more of the protecting groups mentioned hereinabove under “protecting groups”. The protecting groups are then wholly or partly removed according to one of the methods described there.

Salts of a compound of formula I with a salt-forming group may be prepared in a manner known per se. Acid addition salts of compounds of formula I may thus be obtained by treatment with an acid or with a suitable anion exchange reagent. A salt with two acid molecules (for example a dihalogenide of a compound of formula I) may also be converted into a salt with one acid molecule per compound (for example a monohalogenide); this may be done by heating to a melt, or for example by heating as a solid under a high vacuum at elevated temperature, for example from 130 to 170° C., one molecule of the acid being expelled per molecule of a compound of formula I.

Salts can usually be converted to free compounds, e.g. by treating with suitable basic compounds, for example with alkali metal carbonates, alkali metal hydrogencarbonates, or alkali metal hydroxides, typically potassium carbonate or sodium hydroxide.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

It should be emphasized that reactions analogous to the conversions mentioned in this chapter may also take place at the level of appropriate intermediates (and are thus useful in the preparation of corresponding starting materials).

### Starting Materials

The starting materials of the formulae II, as well as other starting materials (including intermediate) mentioned herein, e.g. below, can be prepared according to or in analogy to methods that are known in the art, are known in the art and/or are commercially available. Novel starting materials, as well as processes for the preparation thereof, are likewise an embodiment of the present invention. In the preferred embodiments, such starting materials are used and the reactions chosen are selected so as to enable the preferred compounds to be obtained.

In the synthesis of starting materials, the symbols R¹, R², R³, R⁴, R⁵, X, Y, p, q and r in the formulae given in the starting materials and intermediates given below have the meanings given for a compound of the formula I or as indicated specifically, while A is as defined for a compound of the formula II or as indicated specifically.

A compound of the formula II is, for example, prepared by reacting a compound of the formula III,

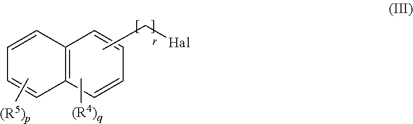

wherein Hal is halo, especially chloro or bromo, or is lower alkanesulfonyl, such as methansulfonyl, with an acetyl salicylic acid ester of the formula IV,

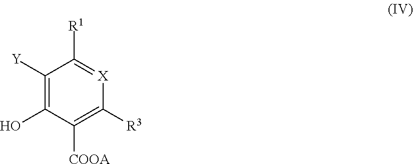

e.g. in the presence of a base, such as an alkali metal carbonate, e.g. potassium carbonate, and optionally of an alkali metal iodide, e.g. potassium iodide, in an appropriate solvent, such as an N,N-di-lower alkyl-lower alkanamide, e.g. dimethylformamide, at temperatures e.g. in the range from 20° C. to the boiling point of the reaction mixture, e.g. from 20 to 80° C.; or using alternative conditions appropriate for substitution, e.g. an alkali metal hydride, especially sodium hydride, in an appropriate solvent, e.g. as just mentioned, at lower temperatures, e.g. from −80 to 20° C. Alternatively, a compound analogous to that of the formula III wherein instead of Hal hydroxyl is present can be used as a starting material and the reaction can take place as described above in the presence of an alkali metal halogenide, especially potassium iodide, or by first forming the C₁-C₇-alkane (e.g. methane)-sulfonate of the formula III by reacting the corresponding C₁-C₇-alkanesulfonyl halogenide (e.g. chloride) in the presence of a tertiary nitrogen base, e.g. a tri-(C₁-C₇-alkyl)amine, in an appropriate solvent, e.g. toluene, for example at temperatures in the range from −20 to 50° C., e.g. at about room temperature,

In a compound of the formula II wherein R¹ is halo, especially iodo, bromo or chloro, this halo may be replaced (especially under Suzuki-(Miyaura) conditions, that is, by palladium-catalyzed crosscoupling of organoboranes) by reacting the halo-R¹-carrying compound of the formula II with a compound of the formula (V)

R¹*-D (V)

wherein R¹* is unsubstituted or substituted aryl, unsubstituted or substituted alkenyl or unsubstituted or substituted heteroaryl, each bound to D via a carbon atom, as defined for R¹ in a compound of the formula I, and D is —B(OH)₂ or is a group of the formula

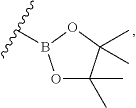

preferably under the conditions of a Suzuki-reaction, preferably in a mixture of a polar aprotic solvent, such as dimethylformamide (DMF) or tetrahydrofuran, and water in the presence of a catalyst for the cross-coupling, especially a noble metal catalyst, preferably a palladium catalyst, such as palladium(II) complex, for example bis(triphenylphosphine)palladium (II) dichloride, in the presence of a base, such as potassium carbonate, sodium hydroxide or sodium carbonate, at a preferred temperature in the range from 60° C. to 130° C., e.g. at about 80° C.; or according to a another preferred method in a cyclic ether solvent, e.g. tetrahydrofuran, in the presence of a catalyst for the cross coupling, especially a noble metal catalyst, preferably a palladium (0) complex, for example tris(dibenzylideneacetone)-dipalladium(0), in the presence of an appropriate ligand, such as 2-dicyclohexylphosphino-2′,6′-dimethoxybiphenyl (SPhos), at a preferred temperature in the range from 60 to 150° C.; if required conducting the reaction in a sealed vessel (e.g. a seal reactor) if the boiling point of the reaction mixture is exceeded and especially if the heating is effected by microwave excitation, thus yielding a corresponding compound of the formula II wherein R¹ is unsubstituted or substituted aryl, unsubstituted or substituted alkenyl or unsubstituted or substituted heteroaryl, each bound to the rest of the molecule via a carbon atom.

In a compound of the formula II wherein R¹ is halo, said halo may be replaced with an N-bound unsubstituted or substituted heterocyclyl or with NR₂ as defined for a compound of the formula I, respectively, by reaction with a compound of the formula (VI)

R¹**—H (VI)

wherein R¹** is unsubstituted or substituted heterocyclyl or NR₂, each bound via nitrogen to the hydrogen atom in formula VI, e.g. in the presence of a palladium(II) catalyst, such as Pd(OAc)₂, and of (racemic) 2,2′-bis(diphenylphosphino)-1,1′-binaphthyl [(rac)-BINAP)], and a base, especially an alkali metal carbonate, e.g. cesium carbonate, in an appropriate solvent, such as a cyclic ether, e.g. dioxane, at preferred temperatures in the range from 30° C. to the boiling temperature of the reaction mixture, e.g. in the range from 70 to 110° C., yielding the corresponding compound(s) of the formula II. Alternatively, the reaction can take place in the presence of a palladium(0) catalyst, such as tris(dibenzylideneacetone)dipalladium(0) and 2-dicyclohexylphosphino-2′-(N,N-dimethylamino)biphenyl in an appropriate solvent, e.g. an ether, such as dimethoxyethane, and a base, such as a phosphate salt, e.g. potassium phosphate, at temperatures e.g. in the range from 30 to 100° C., e.g. at 90° C.

A compound of the formula IV can, for example, be obtained by reacting a corresponding salicylic acid of the formula VII,

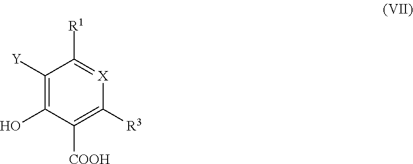

with an alcohol of the formula VIII,

H-A (VIII)

wherein A is as defined for a compound of the formula IV, e.g. methyl or tert-butyl, e.g. in the form of a corresponding acetyl in the presence of an appropriate solvent or solvent mixture, e.g. dimethylformamide and/or toluene, at temperatures e.g. in the range from 30 to 90° C.

In a starting material of the formula II wherein one or more of R⁴ and R⁵ is present and at least one of them is esterified, e.g. with lower alkyl, such as methyl, the alcohol radical, e.g. methyl, may be removed by hydrolysis, e.g. with HCl in dioxane, thus yielding a corresponding compound with a carboxy group instead of the esterified carboxy group(s).

In a compound of the formula II thus obtainable wherein one or more carboxy groups R⁴ and/or R⁵ are present, a carboxy may be converted into a carbamoyl or N-mono- or N,N-disubstituted carbamoyl by reaction first for activation of the carboxy group, e.g. first with ethylchloroformate in the presence of a tertiary nitrogen base, such as triethylamine, in an appropriate solvent, e.g. tetrahydrofuran, e.g. at temperatures in the range from −50 to 20° C., e-g- at about −5° C., or with ethyl-3-(3-dimethylaminopropyl)-carbodiimide and 1-hydroxy-1H-benzotriazole in an appropriate solvent, e.g. dimethylformamide in the presence of a tertiary nitrogen base, e.g. as just mentioned, or with an other coupling agent, followed by reaction with ammonia or a corresponding mono- or disubstituted ammonia in an appropriate solvent, such as water, e.g. at elevated temperatures from 30 to 80° C., thus yielding a corresponding unsubstituted or N-mono- or N,N-disubstituted carbamoyl compound of the formula II.

Alternatively, in a compound of the formula II wherein one or more cyano groups R⁴ and/or R⁵ are present, a cyano may be converted into carbamoyl e.g. by reaction with acetamide in the presence of a catalyst, such as palladium dichloride, in an appropriate solvent, such as tetrahydrofuran and/or water, at temperatures e.g. from 0 to 50° C.

A compound of the formula II wherein one or more moieties hydroxy R⁴ and/or R⁵ are present can be obtained from a precursor wherein instead of the hydroxy a protected hydroxyl is present, e.g. [2-(trimethylsilyl)-ethoxy]-methoxy, by deprotection, e.g. using hydrochloric acid in an appropriate solvent, such as dioxane, at temperatures e.g. in the range from 40 to 80° C.

A compound of the formula III, or an analogue wherein instead of Hal a hydroxyl group is present, can be prepared by reducing an aldehyde of the formula IX,

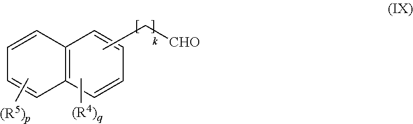

wherein k is 0 or 1 by reduction e.g. with sodium borohydride in an alcohol, e.g. methanol, e.g. at temperatures from −30 to 50° C. For this reaction, if present, a hydroxyl substitutent R⁴ and/or R⁵ can be protected by introduction of a hydroxyl protecting group, e.g. by reaction with 2-(trimethylsilyl)-ethoxy-methoxychloride in an appropriate solvent such as methylene dichloride and in the presence of a tertiary nitrogen base, e.g. N,N-diisopropyl-Nethylamine.

A compound of the formula II wherein R¹ is aryl (e.g. phenyl) substituted by aminoalkyl (especially aminomethyl) may be obtained from a corresponding compound wherein the aryl is substituted by azidoalkyl (especially azidomethyl) by reducing the azido group in the presence of an appropriate reductant, such as polymer supported triphenylphosphine, in an appropriate solvent, e.g. tetrahydrofuran and/or water, at temperatures e.g. from 0 to 50° C. The azidoalkyl (especially azidomethyl) substituent may be formed from a compound of the formula II wherein the eryl substituent is alkyl with one CH₂ group less than in the corresponding azidoalkyl carrying a CHO group by first reducing with e.g. sodium borohydride in an alcohol, such as methanol, e.g. at −30 to 30° C., then activating the hydroxyl on the resulting hydroxyl group, e.g. with methanesulfonyl chloride or toluenesulfonyl chloride in the presence of a tertiary nitrogen base, e.g. N,N-diisopropyl-N-ethylamine, and finally substituting the activated hydroxyl group by reaction with an azide salt, e.g. sodium azide, in an appropriate solvent, e.g. dimethylformamide, for example at temperatures from 0 to 50° C. The aminoalkyl group can then be substituted to give N-mono- or N,N-disubstituted amino-alkyl, e.g. with appropriate alkyl halogenides, appropriate acid halogenides, or the like, under customary reaction conditions.

### Pharmacological Activities

The activity of the compounds of the present invention as FPPS inhibitors can be tested using the scintillation proximity principal similar to a previously reported fatty acid synthase assay using a phospholipid-coated flashplate (see Weiss D R, Glickman J F (2003) Characterization of Fatty Acid Synthase Activity Using Scintillation Proximity. Assay and Drug Development Technologies; 1 (1-2):161-6).

Abreviations used: SPA Scintillation Proximity Assay
FPPS Farnesyl pyrophosphate synthase
FPP Farnesyl pyrophosphate
IPP Isopentenyl pyrophosphate
GPP Geranyl pyrophosphate
DMAPP Dimethyl allyl pyrophosphate
FlashPlate™ Scintillating microtiter plate

Prior FPPS assay methods have used organic:aqueous extraction to separate substrate from product. These methods are extremely time consuming and not compatible with testing large numbers (greater than 20,000) compounds.

The FlashPlate method described herein has the advantages of enabling the rapid testing of large numbers of compounds, easily, and directly. The product formation can be detected by using a phospholipid-coated “Flashplate” (trademark, Perkin-Elmer Lifesciences) which comprises surface-embedded scintillation materials. The lipophilic tritiated FPP which is formed binds to the plate while the tritiated IPP does not. The radiolabelled lipophilic product of the reaction is thus captured on the “Image FlashPlate” which emits photons when tritium is in close proximity. Additionally the use of the LEADseeker imager General Electric, Amersham Lifesciences Division, Cardiff, G B is incorporated which has distinct advantages in plate reading time and in reduced compound interference from yellow compounds over the previously cited Fatty Acid synthase assay. (Weiss Glickman 2003).

All steady-state kinetic parameters are determined by fitting to the Henri-MichaelisMenten equation using the non-linear regression algorithm of GraphPad Prism software (GraphPad Prism version 4.00 for Windows, GraphPad Software, San Diego Calif. USA),

V=Vₘₐₓ[S]/[S]+Kₘ

where
Vₘₐₓ equals the maximal rate of product formation over time;
[S]=the concentration of IPP or GPP;
Kₘ=the Henri-Michaelis-Menten constant which is includes factors for affinity and catalytic rate.
K_{cat} is determined by Vₘₐₓ/[FPPS]
IC₅₀ curves are fit to a variable slope, sigmoidal curve using non-linear regression algorithm in GraphPad Prism software as

Y=bottom+(top-bottom)/1+10^{(log IC50-X)×Hill Slope}.

### Materials

Recombinant human Farnesyl Pyrophosphate synthase (FPPS) was cloned, expressed and purified as previously described {J.-M. Rondeau et al., ChemMedChem 2006, 1, 267-271.} and stored as a 10 mg/mL stock solution in 25 mM Tris pH 7.4, 25 mM NaCl, 2 mM DTT (dithiothreitol). Geranyl pyrophosphate (GPP) was purchased from Anawa AG (Switzerland) and stored as a 1 mg/mL solution in 4 parts isopropanol:3 parts ammonia: 1 part water. 1-[³H]Isopentenyl pyrophosphate (IPP), 50 Ci/mmol; 1 Ci/mL, was purchased from Anawa AG and stored in ethanol:ammonia hydroxide 1:1 at −80 C. 1-[³H] Farnesyl pyrophosphate triammonium salt, 100 Ci/mmol; 1 mCi/mL in 70% ethanol, 0.25 M ammonium bicarbonate was purchased from Anawa AG. Phospholipid-coated 384-well image FlashPlates™ were purchased from PerkinElmer. the assay buffer consisted of 20 mM HEPES pH7.4, 5 mM MgCl₂ and 1 mM CaCl₂.

The FPPS assay is performed in a final detection volume of 12 μl under steady-state conditions as follows:

To the lipid-coated flashplate, note: LEADseeker (trademark) should be spelled consistently. FlashPlates (trademark) should be spelled consistently throughout.

3 μl of test compound solution in 18% DMSO/water or 18% DMSO/assay buffer (carrier control) (end concentration of DMSO in the assay 4.5%),

3 μl of GPP working solution, final concentration 150 nM

3 μl of [³H]-IPP working solution final concentration 150 nM

3 μl of FPPS working solution are added, final concentration 500 μM.

All components are diluted in assay buffer. After addition of all components (in the order listed above), the mixture is incubated for 45 minutes at room temperature.

The inhibition of the FPPS enzymatic reaction by compounds is measured, in a LEADseeker IV (Amersham Biotech), reader, reading time 2 min, method SPA, using for flat field correction the Amersham 384-well standard and quasi-coincident radiation correction, is used.

Test compounds are arrayed in an 8 or 16 point, 2 or 3-fold serial dilution series in 90% DMSO such that the highest concentration is 2 mM in 90% DMSO. In order to obtain replicate data, these compound source plates are diluted and replicated into 384 well image FlashPlates (using a CyBiWell HTS pipetter) to contain 3 μL of compound solution each, to which the assay reagents are added and read. This procedure results in a dose response curve performed in triplicate with 100 μM being the highest concentration tested.

As positive control, Zometa can be used, which inhibits the reaction with an 1050 of between 50 and 200 nM.

Compounds of the formula I can be shown in this test system (called FPPS SPA in the Examples) to have IC₅₀ values for inhibition in the range from 50 nM to 100 μM, preferably from 50 nM to 20 μM.

Due to their ability to inhibit FPPS, and thus on the one hand cholesterol biosynthesis, on the other hand protein farnesylation, the compounds of the formula I are, inter alia, useful in the treatment or in the manufacture of pharmaceutical preparations for the treatment of cholesterol biosynthesis related disorders, e.g. for the lowering of the cholesterol level in blood, on the one hand, and/or protein farnesylation related disorders on the other hand, especially proliferative diseases such as cancer or tumor diseases. Metastasis, especially also bone metastasis, of any cancer or tumor disease is to be included especially. A compound of the formula I may also be used to diminish the susceptibility to cholera toxin by diminishing the number of membrane bound Gₛ protein molecules and for the treatment of pertussis toxin induced coughing by diminishing the number of G proteins. All these disorders are referred to as FPPS-dependent diseases hereinafter (the plural also including the singular, i.e. only one disease).

Where subsequently or above the term “use” is mentioned (as verb or noun) (relating to the use of a compound of the formula I or a pharmaceutically acceptable salt thereof and comparable embodiments of the invention like methods of their use and the like), this includes any one or more of the following embodiments of the invention, respectively: the use in the treatment of an FPPS-dependent disease, the use for the manufacture of pharmaceutical compositions for use in the treatment of an FPPS-dependent disease, methods of use of one or more compounds of the formula I in the treatment of an FPPS-dependent disease, the use of, the use of pharmaceutical preparations comprising one or more compounds of the formula I for the treatment of an FPPS-dependent disease, a process for the manufacture of a pharmaceutical preparation for the treatment of an FPPS-dependent disease, preferably also comprising making it ready for use in such treatment (e.g. adding an instruction insert (e.g. package leaflet or the like), formulation, appropriate preparation, adaptation for specific uses, customizing and the like), and the use of a compound of the formula I for such preparation, and/or all other prophylactic or therapeutic uses mentioned hereinbefore or below, a method of treatment comprising administering a compound of the formula I for the treatment of an FPPS-dependent disease and one or more compounds of the formula I for use in the treatment of a protein kinase dependent disease, as appropriate and expedient and if not stated otherwise. In particular, diseases to be treated and are thus preferred for “use” of a compound of formula I are selected from FPPS-dependent disease (“dependent” meaning dependent “on the activity of”, but also “supported”, not only “solely dependent”, e.g. in case where the FPPS activity is inadequate absolutely or in a given physiological context, either directly or indirectly due to other (e.g. preceding) regulatory mechanisms) diseases mentioned herein, especially proliferative diseases mentioned herein.

Based on the property of the compounds of formula I as potent FPPS inhibitors, the compounds of formula I are especially suitable for the treatment of neoplastic diseases such as cancers and tumors (especially solid tumours but also leukemias, benign or especially malignant tumors), e.g. carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, a neoplasia, a neoplasia of epithelial character or lymphomas, as well as myeloma, especially multiple myeloma, myelodysplastic syndrome, AML (acute myeloid leukemia), AMM (angiogenic myeloid metaplasia), mesothelioma, glioma and glioblastoma, or bone cancer.

On the other hand, compounds of the formula I are especially appropriate for treating cholesterol biosynthesis related disorders, e.g. for the lowering of the cholesterol level in blood, for example for the treatment (including prophylaxis) of atherosclerosis, bilestones, especially cholelithiasis, lipocalcinogranulomatosis, hypercholesterolaemia, hyperlipoproteinaemia, cholesterol crystal embolism, myocardial infection, cerebral infarction, angina pectoris, and/or the like, also as auxiliary treatment together with other treatment (Including prophylactic) measures.

Furthermore, in view of the activities disclosed herein, the compounds of the formula I are especially appropriate for treating in general or inflammation related types of bone loss, including osteoporose, arthritis including rheumatoid arthritis, osteoarthritis and Paget's Disease.

### Pharmaceutical Methods, Preparations and the Like

Where in the following reference is made to compounds of the formula I, the novel compounds of that formula are especially preferred.

The invention relates also to pharmaceutical compositions comprising a compound of formula I, to their use in the therapeutic (in a broader aspect of the invention also prophylactic) treatment or a method of treatment of an FPPS-dependent disease, especially the preferred diseases mentioned above, to the compounds for said use and to pharmaceutical preparations and their manufacture, especially for said uses, and to methods of use of a compound of the formula I in the treatment of such a disease.

The present invention also relates to pro-drugs of a compound of formula I that convert in vivo to the compound of formula I as such. Any reference to a compound of formula I is therefore to be understood as referring also to the corresponding pro-drugs of the compound of formula I, as appropriate and expedient.

The pharmacologically acceptable compounds of the present invention may be present in or employed, for example, for the preparation of pharmaceutical compositions that comprise an effective amount of a compound of the formula I, or a pharmaceutically acceptable salt thereof, as active ingredient together or in admixture with one or more inorganic or organic, solid or liquid, pharmaceutically acceptable carriers (carrier materials).

The invention relates also to a method of treatment for a disease that responds to inhibition of an FPPS-dependent disease and/or a proliferative disease, which comprises administering a prophylactically or especially therapeutically (against the mentioned diseases) effective amount of a compound of formula I according to the invention, or a tautomer thereof or a pharmaceutically acceptable salt thereof, especially to a warm-blooded animal, for example a human, that, on account of one of the mentioned diseases, requires such treatment.

Furthermore, the invention provides the use of a compound according to the definitions herein, or a pharmaceutically acceptable salt, or a hydrate or solvate thereof for the preparation of a medicament for the treatment of an FPPS-dependent disease, especially a proliferative disease or a cholesterol biosynthesis related disorder.

The invention especially relates to the use of a compound of the formula I (or a pharmaceutical formulation comprising a compound of the formula I) in the treatment of one or more of the diseases mentioned above and below where the disease(s) respond or responds (in a beneficial way, e.g. by partial or complete removal of one or more of its symptoms up to complete cure or remission) to an inhibition of FPPS, especially where FPPS shows (in the context of other regulatory mechanisms) inadequately high or more preferably higher than normal (e.g. constitutive) activity.

A compound of the formula I may also be used to advantage in combination with other anti-proliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; N-bisphosphonic acid derivatives; cathepsin K inhibitors; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array PioPharma, AZD6244 from AstraZeneca, PD181461 from Pfizer, leucovorin, EDG binders, antileukemia compounds, ribonucleotide reductase inhibitors, S-adenosylmethionine decarboxylase inhibitors, antiproliferative antibodies or other chemotherapeutic compounds. Further, alternatively or in addition they may be used in combination with other tumor treatment approaches, including surgery, ionizing radiation, photodynamic therapy, implants, e.g. with corticosteroids, hormones, or they may be used as radiosensitizers. Also, in antiproliferative treatment, combination with anti-inflammatory drugs is included.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, colchicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA). The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cisplatin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, protein tyrosine kinase and/or serine and/or threonine kinase inhibitors or lipid kinase inhibitors, e.g.,
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, e.g. imatinib;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
j) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a P13K inhibitor) or AT7519 (CDK inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr <1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, or CDC25, e.g. okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “N-bisphosphonic acid derivatives” as used herein includes, but is not limited to, 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529. especially etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA. All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The term “cathepsin K inhibitors” as used herein includes, but is not limited to, the compounds exemplified in U.S. Pat. No. 6,353,017B1 and WO 03/020278A1.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase recaptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate.

Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt.

Somatostatin receptor antagonists as used herein refers to compounds which target, treat or inhibit the somatostatin receptor such as octreotide, and SOM230.

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term “EDG binders” as used herein refers a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydrooxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy includes treatment with compounds, such as e.g. VISUDYNE and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as e.g. fluocinolone, dexamethasone.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art, such as in the documents cited above.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

The invention also provides a pharmaceutical preparation, comprising a compound of formula I as defined herein, or an N-oxide or a tautomer thereof, or a pharmaceutically acceptable salt of such a compound, or a hydrate or solvate thereof, and at least one pharmaceutically acceptable carrier.

A compound of formula I can be administered alone or in combination with one or more other therapeutic compounds, possible combination therapy taking the form of fixed combinations or the administration of a compound of the invention and one or more other therapeutic (including prophylactic) compounds being staggered or given independently of one another, or the combined administration of fixed combinations and one or more other therapeutic compounds. A compound of formula I can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

The dosage of the active ingredient (=compound of the formula I in free and/or pharmaceutically acceptable salt form) depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

The dose of a compound of the formula I or a pharmaceutically acceptable salt thereof to be administered to warm-blooded animals, for example humans of approximately 70 kg body weight, is preferably from approximately 3 mg to approximately 10 g, more preferably from approximately 10 mg to approximately 2.5 g per person per day, divided preferably into 1 to 3 single doses which may, for example, be of the same size. Usually, children receive half of the adult dose.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The invention relates also to pharmaceutical compositions comprising an effective amount, especially an amount effective in the treatment of one of the above-mentioned disorders, of a compound of formula I or an N-oxide or a tautomer thereof together with one or more pharmaceutically acceptable carriers that are suitable for topical, enteral, for example oral or rectal, or parenteral administration and that may be inorganic or organic, solid or liquid.

There can be used for oral administration especially tablets or gelatin capsules that comprise the active ingredient together with pharmaceutically acceptable carrier materials, e.g. diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting compounds and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving or lyophilising processes, and comprise approximately from 1% to 99%, especially from approximately 1% to approximately 20%, active ingredient(s).

Additionally, the present invention provides a compound of formula I, or a pharmaceutically acceptable salt of such a compound, for use in a method for the treatment of the human or animal body, especially for the treatment of a disease mentioned herein, most especially in a patient requiring such treatment.

The present invention also relates to the use of a compound of formula I, or a pharmaceutically acceptable salt of such a compound, for the preparation of a medicament for the treatment of a proliferative disease.

Furthermore, the invention relates to a method for the treatment of a proliferative disease which responds to an inhibition of FPPS, which comprises administering a compound of formula I or a pharmaceutically acceptable salt thereof, wherein the radicals and symbols have the meanings as defined above, especially in a quantity effective against said disease, to a warm-blooded animal requiring such treatment.

Furthermore, the invention relates to a pharmaceutical composition for treatment of solid or liquid tumours in warm-blooded animals, including humans, comprising an antitumor effective dose of a compound of the formula I as described above or a pharmaceutically acceptable salt of such a compound together with a pharmaceutical carrier.

### EXAMPLES

The following examples serve to illustrate the invention without limiting its scope:

If not indicated otherwise, reactions are conducted at room temperature. Temperatures are given in degrees Celsius (° C.). Ratios e.g. of solvents or eluents in mixtures and the like are given as volume by volume (v/v) ratios. Where the term “heated at” is used, this means “heated to and kept at”.

The following abbreviations are used:
Ac acetyl
BINAP 2,2′-bis(diphenylphosphino)-1,1′-binaphthyl
brine sodium chloride solution saturated at room temperature
Celite® filtering aid based on diatomaceous earth (Celite Corp., Lompoc, Calif., USA)
DMF N,N-dimethylformamide
ES-MS Electrospray Mass Spectrometry
Et ethyl
h hour(s)
HPLC High Performance (or Pressure) Liquid Chromatography
LC-MS Liquid Chromatography-Mass Spectrometry
Me methyl
MS Mass Spectrometry
min minute(s)
Ph phenyl
PTFA polytetrafluoroethylene
TFA trifluoroacetic acid
THF tetrahydrofuran
t_{Ret} retention time

Foot Notes for Examples 1 to 11

HPLC conditions for t_{Ret}:

### Examples 1-8, 10

Method A: LC-MS Waters 2795; column: Sunfire C18; 4.6×20 mm, 3.5 μm; water+0.1% TFA-acetonitrile (AN)+0.1% TFA, 3 ml/min; 40° C.; 4 min 5-100% AN

### Example 11-12, 17

Method B: LC-MS Waters, LCZ single quad MS; column: Waters XTerra C18; 3.0×30 mm, 2.5 μm; (95% water+5% AN+0.2% HCOOH)-100% AN+0.2% HCOOH, 0.6 ml/min; 50° C.; 1.5 min 5-95% AN

### Example 9

Method C: LC-MS HP-1100 (Hewlett-Packard); colomn: Zorbax SB-C18; 3×30 mm, 1.89 μm; water-acetonitrile (AN), 0.7 ml/min, 35° C.; 3.25 min 40-100% AN, 0.75 min 100% AN, 0.25 min 100-40% AN

Foot Notes for Examples 13-16

PTFA: Polytetrafluoroethylene

rac-BINAP: racemic mixture of 2,2′-bis(diphenylphosphino)-1,1′-binaphthalene

### Example 13 and 14

Method D:

Waters chromatographic system with Micromass ZQ MS detection. Aqueous acetonitrile of the following composition containing 0.1% of trifluoroacetic acid is used as a mobile phase at a flow rate of 100 ml/min using a Macherey Nagel Nucleodur 100-10 C-18 column (250×40 mm, 10 μm particle size): isocratic elution for 1 min. at 10% aqueous acetonitrile followed by a linear gradient of 2.0 minutes from 10% aqueous acetonitrile to 40% aqueous acetonitrile followed by a linear gradient of 12.0 minutes from 40% aqueous acetonitrile to 95% aqueous acetonitrile followed by a linear gradient of 2.0 minute from 95% aqueous acetonitrile to 100% acetonitrile. The collection of products is triggered by the MS signal.

### Example 15, 16

15aa-ai, 15ak-ay, 15ba, 16a-c Method D: analytical method 3 minutes

Agilent 1100LC chromatographic system with Micromass ZMD MS detection. A binary gradient composed of A (water containing 5% acetonitrile and 0.2% formic acid) and B (acetonitrile containing 0.2% formic acid) is used as a mobile phase at a flow rate of 0.7 ml/min using a Waters X Terra™ C-18 column (30×3 mm, 2.5 μm particle size): linear gradient of 1.5 minutes from 5% of B to 95% of B followed by a isocratic elution of 1.0 minute of 95% of B.

15aj, az Method E analytical method 5 minutes

Agilent 1100LC chromatographic system with Micromass ZMD MS detection. A binary gradient composed of A (water containing 5% acetonitrile and 0.2% formic acid) and B (acetonitrile containing 0.2% formic acid) is used as a mobile phase at a flow rate of 0.7 ml/min using a Waters X Terra™ C-18 column (30×3 mm, 2.5 μm particle size): isocratic elution during 0.5 minutes of 5% of B followed by a linear gradient of 3.0 minutes from 5% to 95% of B followed by an isocratic elution during 1.0 minute of 95% of B.

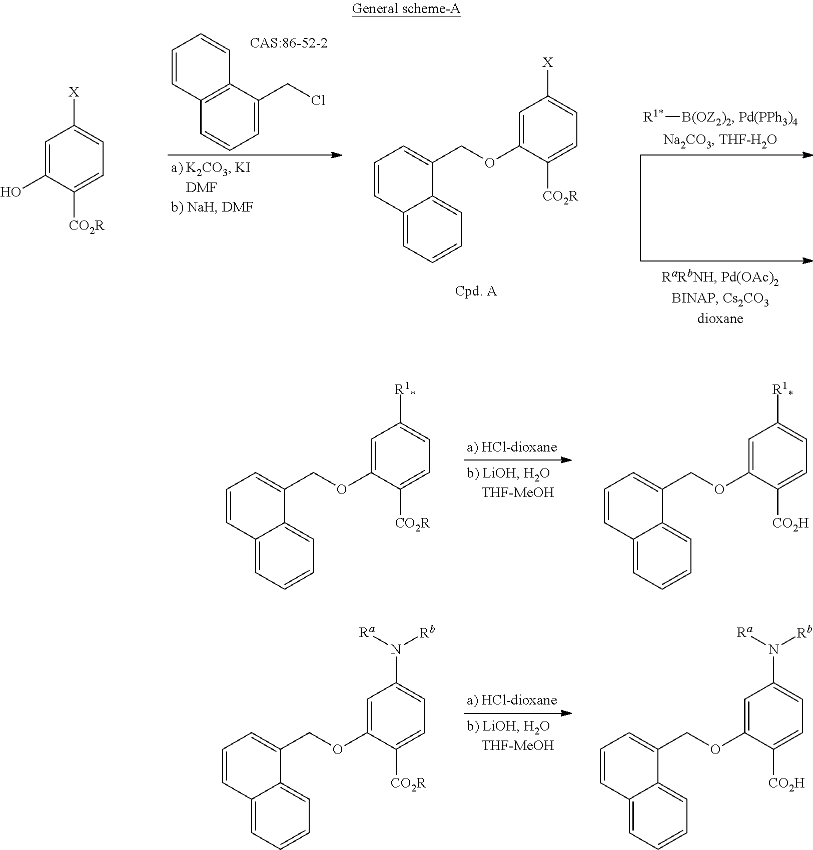

R is alkyl (e.g. methyl or tert-butyl), aryl or arylalkyl; X is hydrogen (then Cpd. A is the product) or halo or trifluoromethanesulfonyl (triflyl); R¹* is unsubstituted or substituted aryl or unsubstituted or substituted heteroaryl bound via a carbon atom to the B, especially as deducible from the Examples; and R^{a} and R^{b} are selected from hydrogen or amino substitutents or together with the nitrogen to which they are bound form a ring, again especially as deducible from the Examples. Z is lower alkyl or BOZ₂ is a group of the formula A,

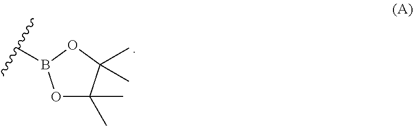

### Example 1

### 4-(2-methyl-phenyl)-2-(naphthalin-1-ylmethoxy)-benzoic acid

To a solution of Educt 1.1 (63 mg, 0.17 mmol) in THF/MeOH (1:1, 2.0 mL), 2M LiOH solution (0.50 mL) is added. The reaction mixture is heated at 60° C. for 1 h. After cooling down to room temperature, the reaction mixture is acidified by 1M HCl solution and extracted with EtOAc. The organic layer is washed with H₂O, dried and concentrated under reduced pressure. The resulting residue is suspended in Et₂O/hexanes to give the title compound, as a white solid after filtration; ES-MS: [M+Na]⁺=391; HPLC: t_{Ret}=3.03 min.

The starting material is prepared as follows:

Stage 1.1: Educt 1.1:

A mixture of Educt 1.2 (100 mg, 0.27 mmol), 2-methylphenylboronic acid (55 mg, 0.41 mmol), 2M Na₂CO₃ solution (0.60 mL, 1.23 mmol) and Pd(PPh₃)₄ (34 mg, 0.03 mmol) in THF (2.4 mL) is heated at 80° C. for 2 h. After cooling down to room temperature, the reaction mixture is diluted with EtOAc. The organic layer is washed with H₂O, dried and concentrated under reduced pressure. The resulting residue is purified by silica gel flash chromatography to give Educt 1.1 as colorless oil; ES-MS: [M+Na]⁺=405: t_{Ret}=3.46 min.

Stage 1.2: Educt 1.2:

A mixture of 4-bromo-2-hydroxybenzoic acid methyl ester (30 g, 130 mmol, CAS:22717-56-2), 1-chloromethylnaphthalene (25 g, 142 mmol, CAS:86-52-2), KI (1.7 g, 10 mmol) and K₂CO₃ (27 g, 195 mmol) in DMF (300 mL) is stirred at 65° C. for 7 h. After cooling down to room temperature, the reaction mixture is diluted with EtOAc. The organic layers are washed with H₂O, dried over MgSO₄ and concentrated under reduced pressure. The resulting residue is suspended in Et₂O/hexanes and stirred at room temperature overnight. Educt 1.2 is obtained as pale yellow solid after filtration; ES-MS: [M+Na]⁺=394: t_{Ret}=3.16 min.

### Example 2

### 4-phenyl-2-(naphthalin-1-ylmethoxy)-benzoic acid

The title compound is synthesized by hydrolysis of Educt 2.1 (58 mg, 0.16 mmol) analogously to the preparation of Example 1. White solid; ES-MS: [M+Na]⁺=377; HPLC: t_{Ret}=2.88 min.

Stage 2.1: Educt 2.1:

Educt 2.1 is synthesized by coupling of Educt 1.2 (100 mg, 0.27 mmol) analogously to the preparation of Educt 1.1. Colorless oil; ES-MS: [M+Na]⁺=391; HPLC: t_{Ret}=3.33 min.

### Example 3

### 4-(2,6-dimethoxy-phenyl)-2-(naphthalin-1-ylmethoxy)-benzoic acid

The title compound of Example 3 is synthesized by hydrolysis of Educt 3.1 (36 mg, 0.084 mmol) analogously to the preparation of Example 1. White solid; ES-MS: [M+Na]⁺=437; HPLC: t_{Ret}=2.66 min.

Stage 3.1: Educt 3.1:

Educt 3.1 is synthesized by coupling of Educt 1.2 (150 mg, 0.40 mmol) analogously to the preparation of Educt 1.1. White solid; ES-MS: [M+Na]⁺=451; HPLC: t_{Ret}=3.13 min.

### Example 4

### 4-(3-aminomethyl-phenyl)-2-(naphthalin-1-ylmethoxy)-benzoic acid

To a solution of Educt 4.1 (80 mg, 0.19 mmol) in THF/H₂O (10:1, 1.1 mL), polymer supported triphenylphosphine (612 mg, 0.95 mmol) is added at room temperature. The reaction mixture is stirred at 60° C. for 1 h. After cooling down to room temperature, the reaction mixture is diluted with THF and filtered. MeOH (2 mL) and 2M LiOH (1 mL) are added to the solution. The reaction mixture is heated at 60° C. for 1 h. After cooling down to room temperature, the solution is acidified by 1M HCl and extracted with EtOAc. The organic layer is washed with H₂O, dried and concentrated under reduced pressure. The resulting residue is purified by reverse phase preparative HPLC (0.1% TFA, CH₃CN/H₂O) to give the title compound 4 after lyophilization. White solid; ES-MS: [M+H]⁺=384; HPLC: t_{Ret}=1.40 min.

Stage 4.1: Educt 4.1:

To a solution of Educt 4.2 (150 mg, 0.38 mmol) in MeOH (2.0 mL), NaBH₄ (16 mg, 0.42 mmol) is added at 0° C. The reaction mixture is stirred at room temperature for 3 h. After treatment with saturated NH₄Cl solution, the mixture is extracted with EtOAc. The organic layer is dried and concentrated under reduced pressure. The resulting residue is dissolved in toluene (1.0 mL) without purification. To the reaction mixture, diisopropylethylamine (63 mg, 0.49 mmol) and methanesulfonyl chloride (65 mg, 0.57 mmol) are added. After stirring at room temperature for 3 h, the reaction mixture is diluted with EtOAc. The organic layer is washed with H₂O, dried and concentrated under reduced pressure. To a solution of the resulting residue in DMF (3.0 mL), NaN₃ (74 mg, 1.14 mmol) is added. After stirring at room temperature overnight, the reaction mixture is diluted with EtOAc. The organic layer is washed with H₂O, dried and concentrated under reduced pressure. The resulting residue is purified by silica gel flash chromatography to give Educt 4.1 as colorless oil; ES-MS: [M+Na]⁺=446: t_{Ret}=3.37 min.

Stage 4.2: Educt 4.2:

Educt 4.2 is synthesized by coupling of Educt 1.2 (500 mg, 1.3 mmol) analogously to the preparation of Educt 1.1. Pale yellow solid; ES-MS: [M+Na]⁺=419; HPLC: t_{Ret}=3.08 min.

### Example 5

### 4-(2-oxopyrrolidino)-2-(naphthalin-1-ylmethoxy)-benzoic acid

The title compound 5 is synthesized by hydrolysis of Educt 5.1 (42 mg, 0.11 mmol) analogously to the preparation of Example 1. White solid; ES-MS: [M+Na]⁺=384; HPLC: t_{Ret}=2.17 min.

Stage 5.1: Educt 5.1:

A mixture of Educt 1.2 (100 mg, 0.27 mmol), pyrrolidinone (69 mg, 0.81 mmol), Cs₂CO₃ (130 mg, 0.40 mmol), Pd(OAc)₂ (7.0 mg, 0.030 mmol) and racemic BINAP (30 mg, 0.045 mmol) in dioxane (3.0 mL) is heated at 100° C. under N₂ atmosphere for 3 h. After cooling down to room temperature, the reaction mixture is filtered and washed with EtOAc. The filtrate is concentrated under reduced pressure. The resulting residue is purified by silica gel flash chromatography to give Educt 5.1 as colorless oil; ES-MS: [M+Na]⁺=398: t_{Ret}=2.53 min.

### Example 6

### 4-pyrrolo-2-2-(naphthalin-1-ylmethoxy)-benzoic acid

The title compound 6 is synthesized by hydrolysis of Educt 6.1 (33 mg, 0.090 mmol) analogously to the preparation of Example 1. White solid; ES-MS: [M+Na]⁺=370; HPLC: t_{Ret}=2.72 min.

Stage 6.1: Educt 6.1:

Educt 6.1 is synthesized by coupling of Educt 1.2 (100 mg, 0.27 mmol) analogously to the preparation of Educt 5.1. Colorless oil, ES-MS: [M+H]⁺=362: t_{Ret}=3.16 min.

### Example 7

### 4-piperidino-2-(naphthalin-1-ylmethoxy)-benzoic acid

The title compound 7 is synthesized by hydrolysis of compound of Stage 7.1 (42 mg, 0.11 mmol) analogously to the preparation of compound of Example 1. White solid; ES-MS: [M+H]⁺=362; HPLC: t_{Ret}=2.83 min.

Stage 7.1: Educt 7.1

Educt 7.1 is synthesized by coupling of Educt 1.2 (100 mg, 0.27 mmol) analogously to the preparation of Educt 5.1. Colorless oil; ES-MS: [M+H]⁺=376; HPLC: t_{Ret}=3.26 min.

### Example 8

### 4-phenylamino-2-(naphthalin-1-ylmethoxy)-benzoic acid

The title compound 8 is synthesized by hydrolysis of Educt 8.1 (51 mg, 0.11 mmol) analogously to the preparation of Example 1. White solid; ES-MS: [M+H]⁺=370; HPLC: t_{Ret}=2.67 min.

Stage 8.1: Educt 8.1:

Educt 8.1 is synthesized by coupling of Educt 1.2 (100 mg, 0.27 mmol) analogously to the preparation of Educt 5.1. Colorless oil; ES-MS: [M+Na]⁺=406; HPLC: t_{Ret}=3.06 min.

### Example 9

### 4-(4-carbamoyl-phenyl)-2-(naphthalin-1-ylmethoxy)-benzoic acid

Similar to example 1, Educt 1.1, the reaction of 4-carbamoyl-phenylboronic acid with 4-bromo-2-(naphthalen-1-ylmethoxy)-benzoic acid methyl ester (Educt 1.2) leads to 4′-carbamoyl-3-(naphthalen-1-ylmethoxy)-biphenyl-4-carboxylic acid methyl ester (ES-MS: [M+H]⁺=412: t_{Ret}=2.63 min).

Ester hydrolysis according to example 1 yields the title compound of example 17 as a white solid. (ES-MS: [M+H]⁺=398: t_{Ret}=1.42 min (method C)).

### Example 10

### 2-(5-cyano-napthalin-1-yl-methoxy)-benzoic acid

A mixture of Educt 10.1 (10.0 mg, 0.028 mmol) is heated in 4M HCl-dioxane (2 mL) at 60° C. for 3 h. After cooling down to room temperature, the reaction mixture is diluted with EtOAc. The resulting residue is purified by reverse phase preparative HPLC (0.1% TFA, CH₃CN/H₂O) to give the title compound of Example 10 as a white solid; ES-MS: [M+Na]⁺=326: t_{Ret}=2.23 min.

The starting material is prepared as follows:

Stage 10.1: Educt 10.1

To a solution of 5-(hydroxymethyl)-1-naphthalenecarbonitrile (200 mg, 1.09 mmol, CAS: 176907-25-8) and diisopropylethylamine (206 mg, 1.6 mmol) in toluene, methanesulfonyl chloride (148 mg, 1.3 mmol) is added at room temperature. After stirring at the same temperature for 2 h, the reaction mixture is diluted with EtOAc. The organic layer is washed with H₂O and brine, dried and concentrated under reduced pressure to give pale yellow solids. A mixture of the resulting solids, 2-hydroxybenzoic acid tert-butyl ester (213 mg, 1.1 mmol, CAS: 23408-05-01), K₂CO₃ and KI in DMF (3.0 mL) is stirred at 65° C. for 4 h. After cooling down to room temperature, the reaction mixture is diluted with EtOAc. The organic layer is washed with H₂O, dried and concentrated under reduced pressure. The resulting residue is purified by silica gel flash chromatography to give Educt 10.1 as a white solid; ES-MS: [M+Na]⁺=382: t_{Ret}=2.11 min.

### Example 11

### 4-(2-hydroxyethoxy)-2-(naphthalin-1-ylmethoxy)-benzoic acid

A mixture of 2-hydroxy-4-(2-hydroxy-ethoxy)benzoic acid (101 mg, 0.51 mmol), 1-chloromethylnaphthalene (25 g, 142 mmol, CAS:86-52-2), and 4.5 equivalents of K₂CO₃ (2.3 mmol) in DMF (1.5 mL) is stirred at 150° C. for 16 h. After cooling down to room temperature, 1.33 mL MeOH and 0.254 mL 40% aqueous NaOH are added to the reaction mixture, and the reaction mixture is heated at 95° C. for 2.5 h. After cooling down to room temperature, a precipitate formed is filtered off and the remaining solution is evaporated and then dissolved in 3 mL of H₂O, acidified by 1M HCl solution and extracted with CH₂Cl₂. The organic layer is washed with H₂O, dried and concentrated under reduced pressure. The title compound, Example 11, is obtained as a white solid; ES-MS: [M−H]⁻=337: t_{Ret}=1.45 min.

### Example 12

### 4-carboxymethoxy-2-(naphthalin-1-ylmethoxy)-benzoic acid

The chromic acid oxidizing reagent is prepared by dissolving 534 mg of chromium trioxide in 2 ml of distilled water. To this solution 0.46 ml. of concentrated sulfuric acid is added. To a vigorously agitated solution of 15 mg (44.3 μmole) of the compound of Example 11 in 1 mL of acetone, sufficient chromic acid oxidizing reagent is added to permit the orange color of the reagent to persist.

The cooled pot residue is transferred to a separatory funnel, CH₂Cl₂ is added, and the mixture is extracted with K₂CO₃ solution two times. The organic layer is dried over anhydrous magnesium sulfate, filtered, and the methylene chloride is evaporated. The resulting residue is purified by reversed phase chromatography to give the title compound of Example 20 as a white solid; ES-MS: [M+H]⁺=353: t_{Ret}=1.45 min.

### Example 13

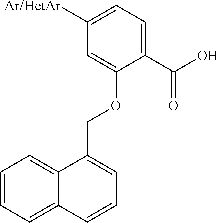

(wherein Ar/HetAr) is aryl or heteroaryl which may be unsubstituted or substituted bound via a ring carbon atom)

Generic name: 4-(Unsubstituted or substituted aryl- or heteroaryl)-2-(naphthalen-1-ylmethoxy)-benzoic acids.

Generic Procedure:

Parallel synthesis of 4-(unsubstituted or substituted aryl- or heteroaryl)-2-(naphthalen-1-ylmethoxy)-benzoic acids

To an array of microwave resistant glass tubes, one of 29 boronic acids (0.256 mmol) is added into each tube. Then 4-bromo-2-(naphthalen-1-ylmethoxy)-benzoic acid (0.213 mmol), a saturated solution of PdCl₂(PPh₃)₂ (0.9 ml) in a mixture dimethoxyethane (7 parts)/ethanol (2 parts)/water (3 parts) and a 2 molar aqueous solution of Na₂CO₃ (0.215 ml) are added to each tube. All tubes are sealed with a pressure resistant aluminium cap, and the reaction mixtures are individually irradiated in a microwave oven until a temperature of 110° C. is reached. This temperature is held constant for 10 min. After cooling to room temperature, the reaction mixtures are individually transferred to 100 ml glass tubes and ethyl acetate (2 ml) and water (15 ml) are added to each tube. After phase separation each tube is extracted 5 times with ethyl acetate (5 ml) followed by evaporation of the combined organic extracts. The resulting array of crude material is transferred into individual microwave resistant glass tubes, followed by the addition in each tube of methanol (0.5 ml), tetrahydrofuran (0.5 ml) and a 1 molar aqueous solution of LiOH (0.5 ml). All tubes are sealed with a pressure resistant aluminium cap and the reaction mixtures are individually irradiated in a microwave oven until a temperature of 120° C. is reached. The temperature is held constant for 12 min and then cooled to room temperature. Acetic acid (0.1 ml) and tetrahydrofuran (2 ml) are individually added to the tubes, followed by filtration of each reaction mixture over a 0.45 μm PTFA membrane. The filtrates are then individually purified by a preparative LC-MS procedure.

Preparative LC-MS Procedure:

Preparative Waters chromatographic system with Micromass® ZQ MS detection (Waters GmbH, Eschborn, Germany). Aqueous acetonitrile of the following composition containing 0.1% of trifluoroacetic acid is used as a mobile phase at a flow rate of 100 ml/min using a Macherey Nagel Nucleodur® 100-10 C-18 column (reversed phase C₁₈-bonded silica; Macherey & Nagel, Duren, Germany; 250×40 mm, 10 μm particle size): isocratic elution for 1 min. at 10% aqueous acetonitrile followed by a linear gradient of 2.0 min from 10% aqueous acetonitrile to 40% aqueous acetonitrile followed by a linear gradient of 12.0 min from 40% aqueous acetonitrile to 95% aqueous acetonitrile followed by a linear gradient of 2.0 minute from 95% aqueous acetonitrile to 100% acetonitrile. The collection of products is triggered by the MS signal.

This generic procedure is used to prepare the following compounds:

| | | IC₅₀ | | Detected |
|---|---|---|---|---|
| Example | Formula | [umol I-1] | Rt [min] | mass |
| 13 aa) | 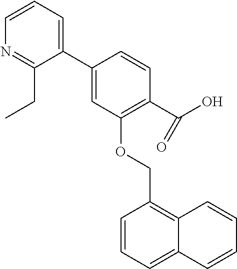 | 0.21 | 7.31 | 386 (MH⁺) |
| 13 ab) | 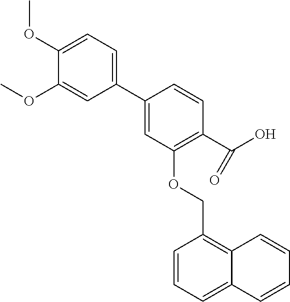 | 0.26 | 7.20 | 437 (M + Na⁺) |
| 13 ac) | 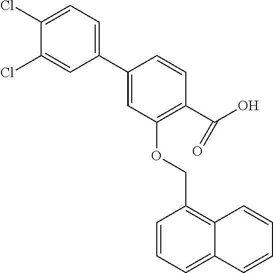 | 1.46 | 10.95 | 446 (M + Na⁺) |
| 13 ad) | 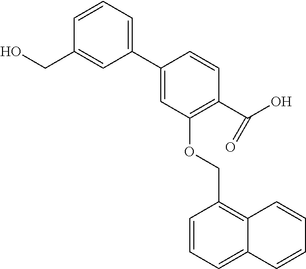 | 0.50 | 6.08 | 407 (M + Na⁺) |
| 13 ae) | 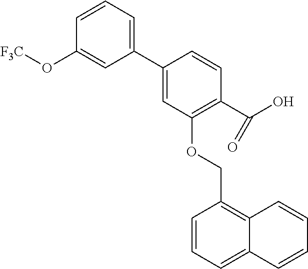 | >10 | 12.83 | 461 (M + Na⁺) |
| 13 af) | 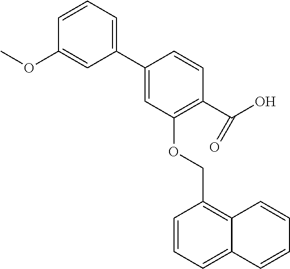 | 1.55 | 8.09 | 407 (M + Na⁺) |
| 13 ag) | 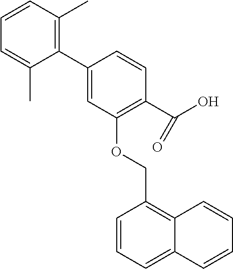 | 1.10 | 9.22 | 405 (M + Na⁺) |
| 13 ah) | 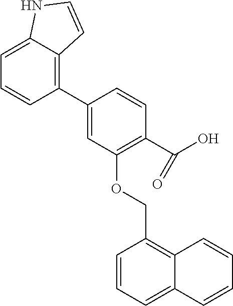 | 0.26 | 6.86 | 415 (M + Na⁺) |
| 13 ai) | 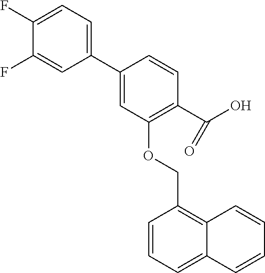 | 0.32 | 8.48 | 413 (M + Na⁺) |
| 13 aj) | 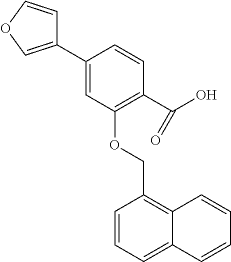 | 0.55 | 7.20 | 367 (M + Na⁺) |
| 13 ak) | 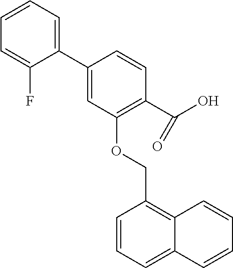 | 0.23 | 7.97 | 395 (M + Na⁺) |
| 13 al) | 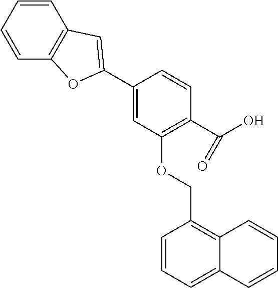 | 1.12 | 9.9 | 417 (M + Na⁺) |
| 13 am) | 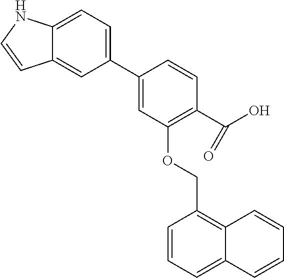 | 0.34 | 7.26 | 415 (M + Na⁺) |
| 13 an) | 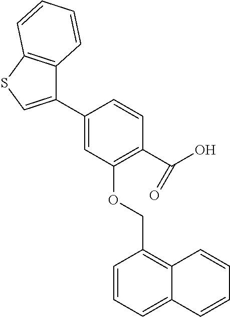 | 1.35 | 9.73 | 433 (M + Na⁺) |
| 13 ao) | 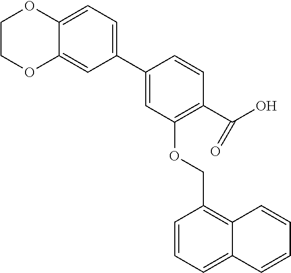 | 0.42 | 7.82 | 435 (M + Na⁺) |
| 13 ap) | 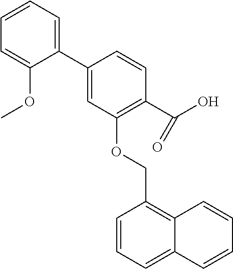 | 1.93 | 8.00 | 407 (M + Na⁺) |
| 13 aq) | 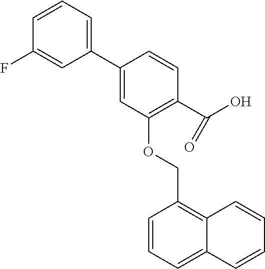 | 1.14 | 8.23 | 395 (M + Na⁺) |
| 13 ar) | 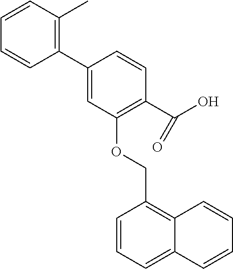 | 0.53 | 8.77 | 391 (M + Na⁺) |
| 13 as) | 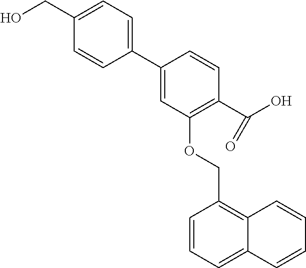 | 0.38 | 6.13 | 407 (M + Na⁺) |
| 13 at) | 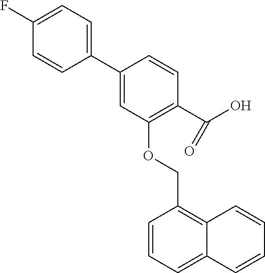 | 0.30 | 8.31 | 395 (M + Na⁺) |
| 13 au) | 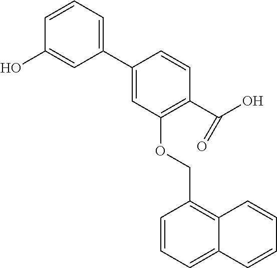 | 0.38 | 6.20 | 393 (M + Na⁺) |
| 13 av) | 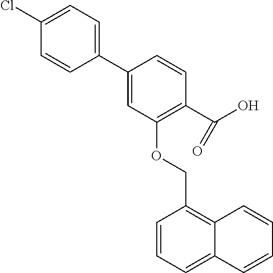 | 0.69 | 9.59 | 411 (M + Na⁺) |
| 13 aw) | 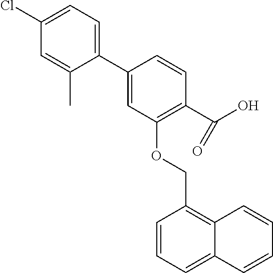 | 0.44 | 10.20 | 425 (M + Na⁺) |
| 13 ax) | 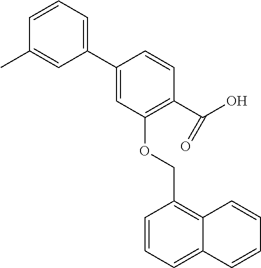 | 0.40 | 9.21 | 391 (M + Na⁺) |
| 13 ay) | 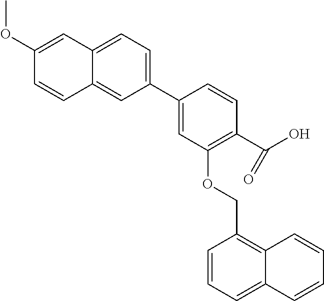 | 0.38 | 10.28 | 457 (M + Na⁺) |
| 13 az) | 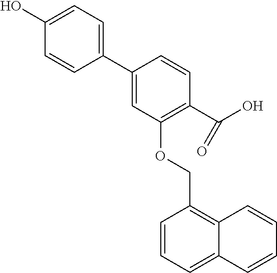 | 0.38 | 6.11 | 393 (M + Na⁺) |
| 13 ba) | 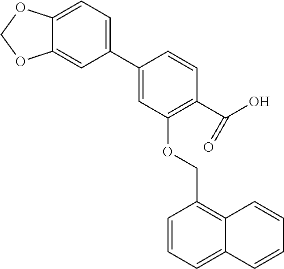 | 0.24 | 8.04 | 421 (M + Na⁺) |
| 13 ca) | 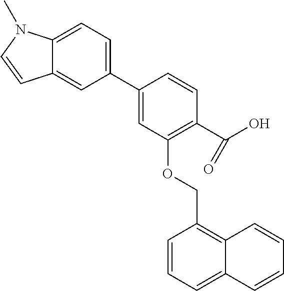 | 0.27 | 8.73 | 430 (M + Na⁺) |
| 13 da) | 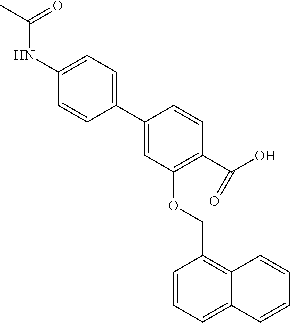 | 0.13 | 6.23 | 434 (M + Na⁺) |

### Example 14

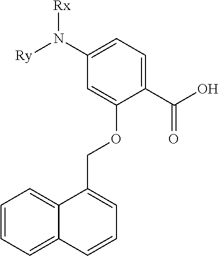 [0272] Rx, Ry=independently of each other as deducible from the individual compounds below.

Generic name: 4-Amino-2-(naphthalen-1-ylmethoxy)-benzoic acid derivatives

Generic Procedure:

### Parallel synthesis of 4-Amino-2-(naphthalen-1-ylmethoxy)-benzoic acids

To an array of microwave resistant glass tubes, one of 20 amines/anilines (0.290 mmol) is added into each tube. Then 4-bromo-2-(naphthalen-1-ylmethoxy)-benzoic acid (0.267 mmol), Cs₂CO₃ (0.373 mmol), rac-BINAP (0.0267 mmol), Pd(OAc)₂ (0.013 mmol) and toluene (1 ml) are added to each tube. All tubes are sealed with a pressure resistant aluminium cap and the reaction mixtures are individually irradiated in a microwave oven until a temperature of 115° C. is reached. This temperature is held constant for 50 min. After cooling to room temperature, the reaction mixtures are individually transferred to 100 ml glass tubes, diluted with water and extracted several times with ethyl acetate. The combined organic extracts are individually evaporated and transferred to an array of microwave resistant glass tubes using methanol (0.4 ml) and tetrahydrofuran (0.4 ml) followed by the addition of a 2 molar aqueous solution of LiOH (0.8 ml) to each tube. All tubes are sealed with a pressure resistant aluminium cap and the reaction mixtures are individually irradiated in a microwave oven until a temperature of 120° C. is reached. The temperature is held constant for 12 min and then cooled to room temperature. Acetic acid (0.2 ml) and methanol (2 ml) are individually added to the tubes, followed by filtration of each reaction mixture over a 0.45 μm PTFA membrane. The filtrates are then individually purified by a preparative LC-MS procedure.

Preparative LC-MS Procedure:

Preparative Waters chromatographic system with Micromass® ZQ MS detection. Aqueous acetonitrile of the following composition containing 0.1% of trifluoroacetic acid is used as a mobile phase at a flow rate of 100 ml/min using a Macherey Nagel Nucleodur® 100-10 C-18 column (250×40 mm, 10 μm particle size): isocratic elution for 1 min. at 10% aqueous acetonitrile followed by a linear gradient of 2.0 min from 10% aqueous acetonitrile to 40% aqueous acetonitrile followed by a linear gradient of 12.0 min from 40% aqueous acetonitrile to 95% aqueous acetonitrile followed by a linear gradient of 2.0 minute from 95% aqueous acetonitrile to 100% acetonitrile. The collection of products is triggered by the MS signal.

This generic procedure is used to prepare the following compounds:

| | | | Detected |
|---|---|---|---|
| Example | Formula | Rt [min] | mass |
| 14 a) | 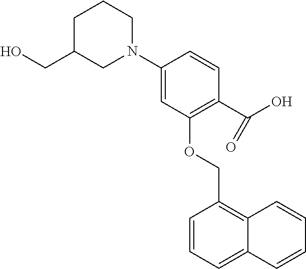 | 5.53 | 392 (MH⁺) |
| 14 b) | 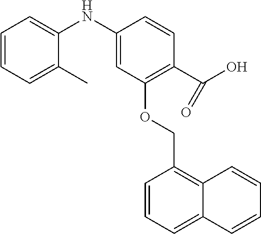 | 7.68 | 406 (M + Na⁺) |
| 14 c) | 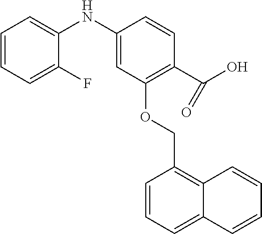 | 7.18 | 410 (M + Na⁺) |
| 14 d) | 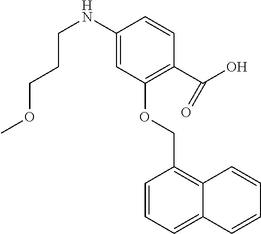 | 6.26 | 366 (MH⁺) |
| 14 e) | 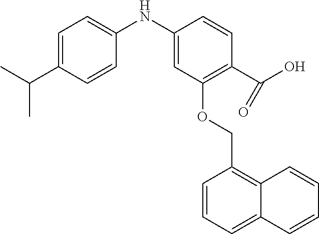 | 6.26 | 366 (MH⁺) |
| 14 f) | 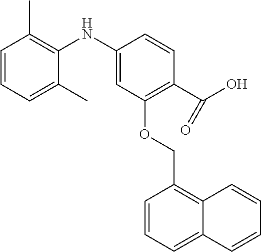 | 7.98 | 420 (M + Na⁺) |
| 14 g) | 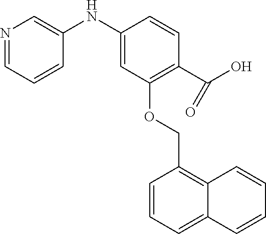 | 4.42 | 371 (MH⁺) |
| 14 h) | 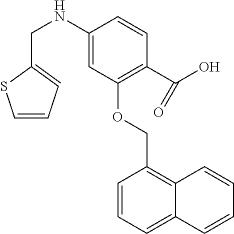 | 6.86 | 390 (MH⁺) |
| 14 i) | 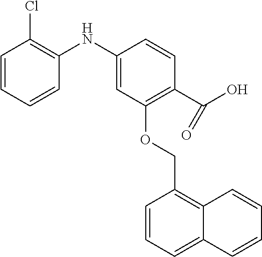 | 7.86 | 426 (M + Na⁺) |
| 14 j) | 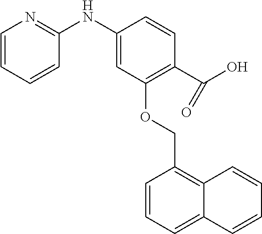 | 4.67 | 371 (MH⁺) |
| 14 k) | 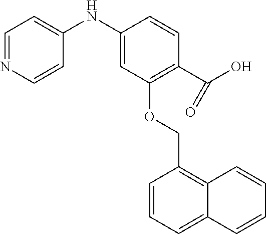 | 4.32 | 371 (MH⁺) |
| 14 l) | 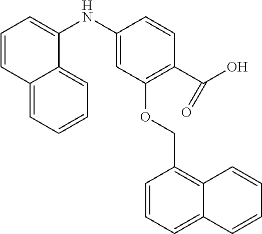 | 8.31 | 420 (MH⁺) |
| 14 m) | 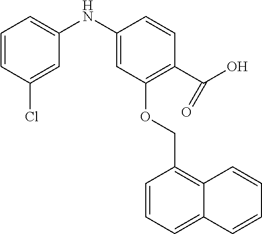 | 8.15 | 426 (M + Na⁺) |
| 14 n) | 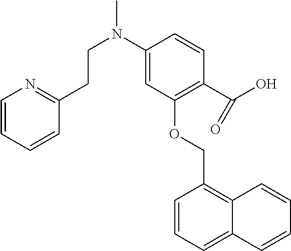 | 4.49 | 413 (MH⁺) |
| 14 o) | 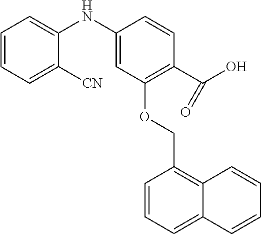 | 6.57 | 417 (M + Na⁺) |
| 14 p) | 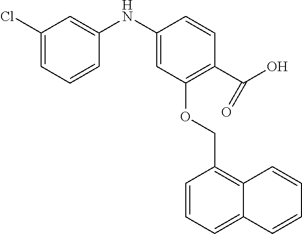 | 9.32 | 418 (MH⁺) |
| 14 q) | 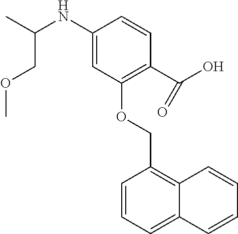 | 6.29 | 366 (MH⁺) |
| 14 r) | 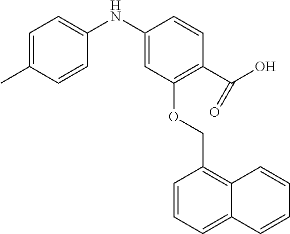 | 7.82 | 406 (M + Na⁺) |
| 14 s) | 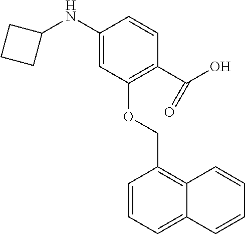 | 7.28 | 370 (M + Na⁺) |
| 14 t) | 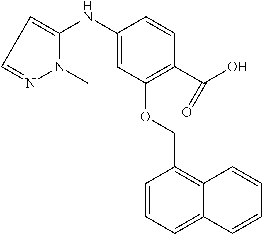 | 5.43 | 374 (MH⁺) |

### Example 15

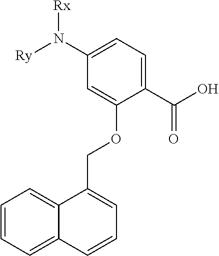

Rx, Ry=independently of each other as deducible from the individual compounds below.

Generic name: 4-Amino-2-(naphthalen-1-ylmethoxy)-benzoic acid derivatives

Generic Procedure:

### Parallel synthesis of 4-Amino-2-(naphthalen-1-ylmethoxy)-benzoic acids

To an array of glass tubes, one of 27 amines/anilines (0.400 mmol) is added into each tube. Then a solution of 4-bromo-2-(naphthalen-1-ylmethoxy)-benzoic acid (0.267 mmol) in 1,2-dimethoxyethane (1.5 ml) and K₃PO₄ (0.800 mmol) is added to each tube. All tubes are flushed with argon and closed. Under argon atmosphere, 2-dicyclohexylphosphino-2′-(N,N-dimethylamino)biphenyl (0.0267 mmol) and tris(dibenzylideneacetone)dipalladium(0) (0.0267 mmol) are added quickly to each tube. The resulting magnetically stirred mixtures are heated under a positive argon atmosphere at 90° C. for 24 hours. After cooling to room temperature, methanol (1 ml) is added to each tube and the reaction mixtures are individually filtered over Celite® 501 and a 0.45 μm PTFA membrane. The filtrates are then individually purified by a preparative LC-MS procedure. The preparative HPLC fractions containing the desired compounds are individually pooled in 100 ml glass vials and solvents are evaporated. Methanol (0.75 ml), tetrahydrofuran (0.75 ml) and a 2 molar aqueous solution of LiOH (0.67 ml) are added to each tube and the tubes are individually closed. The array of reaction mixtures is allowed to stand at 80° C. for 17 hours. After cooling to room temperature, acetic acid (0.058 ml), ethyl acetate (10 ml) and an aqueous phosphate buffer solution at pH 7.0 (10 ml) are added individually to each tube. After phase separation each tube is extracted 3 times with ethyl acetate (10 ml) and the combined organic extracts are dried over MgSO₄ prior to evaporation of the solvent.

Preparative LC-MS Procedure:

Preparative Waters chromatographic system with Micromass® ZQ MS detection. Aqueous acetonitrile of the following composition containing 0.1% of trifluoroacetic acid is used as a mobile phase at a flow rate of 50 ml/min using a Waters Sunfire® C-18 column (reversed phase C₁₈-onded silica, WatersGmbH, Eschborn, Germany; 150×30 mm, 5 μm particle size): isocratic elution for 1 min. at 10% aqueous acetonitrile followed by a linear gradient of 1.5 min from 10% aqueous acetonitrile to 60% aqueous acetonitrile followed by a linear gradient of 7.5 min from 60% aqueous acetonitrile to 95% aqueous acetonitrile followed by a linear gradient of 1.0 minute from 95% aqueous acetonitrile to 100% acetonitrile. The collection of products is triggered by the MS signal.

This generic procedure is used to prepare the following compounds:

| | | IC₅₀ | | Detected |
|---|---|---|---|---|
| Example | Formula | [umol I-1] | Rt [min] | mass |
| 15 aa) | 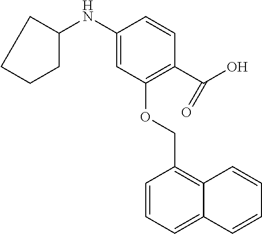 | 0.63 | 1.88 | 362 (MH⁺) |
| 15 ab) | 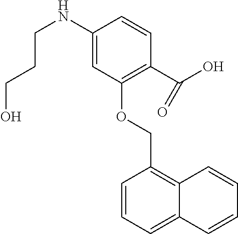 | 2.31 | 1.58 | 352 (MH⁺) |
| 15 ac) | 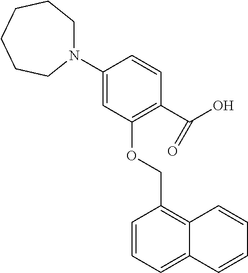 | 0.53 | 1.95 | 376 (MH⁺) |
| 15 ad) | 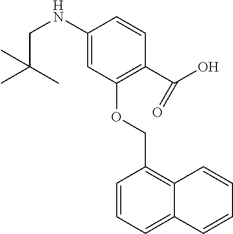 | 0.47 | 1.88 | 364 (MH⁺) |
| 15 ae) | 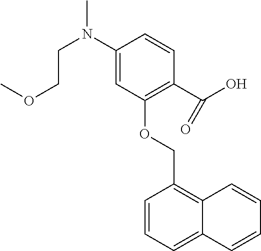 | 1.39 | 1.78 | 366 (MH⁺) |
| 15 af) | 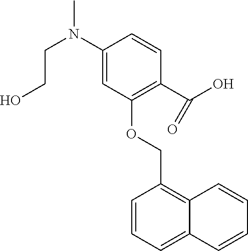 | 0.72 | 1.62 | 352x (MH⁺) |
| 15 ag) | 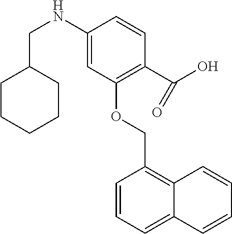 | 2.43 | 2.01 | 390 (MH⁺) |
| 15 ah) | 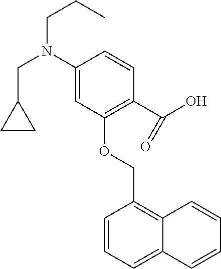 | 1.31 | 2.02 | 390 (MH⁺) |
| 15 ai) | 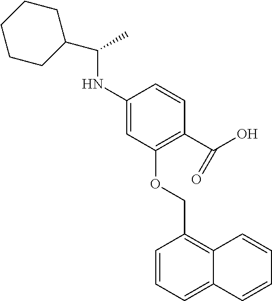 | 3.52 | 2.05 | 404 (MH⁺) |
| 15 aj) | 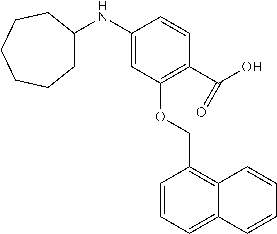 | 1.79 | 3.53 | 390 (MH⁺) |
| 15 ak) | 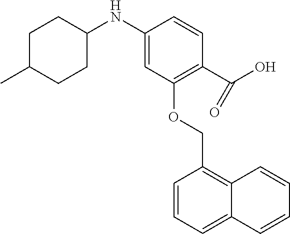 | 1.24 | 1.99 | 390 (MH⁺) |
| 15 al) | 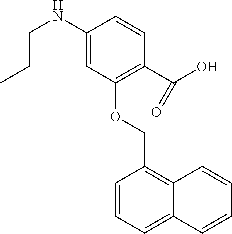 | 0.37 | 1.81 | 336 (MH⁺) |
| 15 am) | 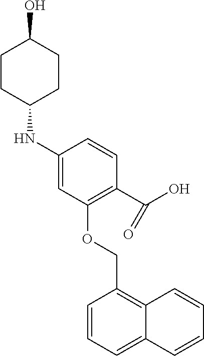 | 0.19 | 1.61 | 392 (MH⁺) |
| 15 an) | 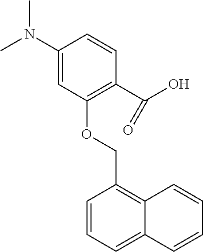 | 0.19 | 1.83 | 322 (MH⁺) |
| 15 ao) | 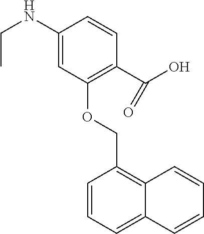 | 0.73 | 1.74 | 322 (MH⁺) |
| 15 ap) | 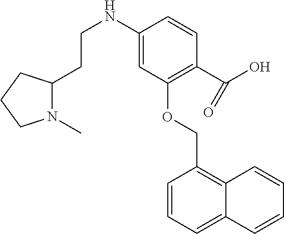 | 17.54 | 1.37 | 405 (MH⁺) |
| 15 aq) | 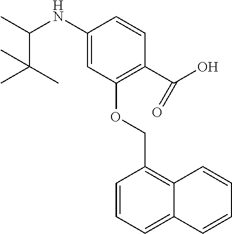 | 0.65 | 1.98 | 378 (MH⁺) |
| 15 ar) | 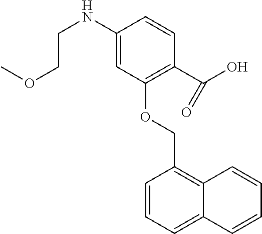 | 8.10 | 1.71 | 352 (MH⁺) |
| 15 as) | 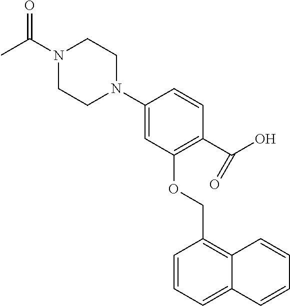 | 6.50 | 1.37 | 405 (MH⁺) |
| 15 at) | 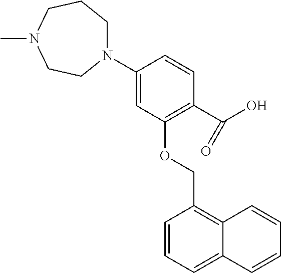 | 6.45 | 1.38 | 391 (MH⁺) |
| 15 au) | 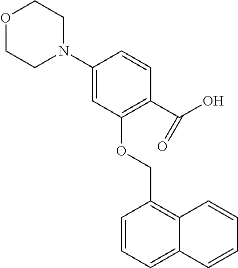 | 3.18 | 1.72 | 364 (MH⁺) |
| 15 av) | 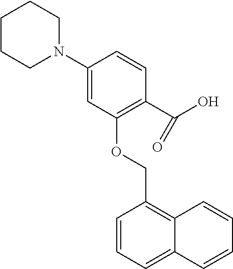 | | 1.81 | 362 (MH⁺) |
| 15 aw) | 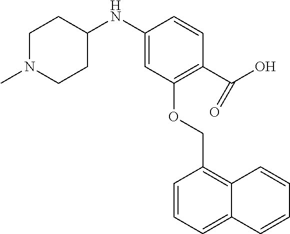 | 4.75 | 1.38 | 391 (MH⁺) |
| 15 ax) | 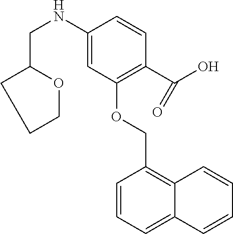 | | 1.75 | 378 (MH⁺) |
| 15 ay) | 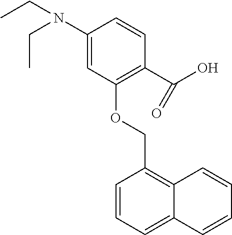 | 43.80 | 1.90 | 364 (MH⁺) |
| 15 az) | 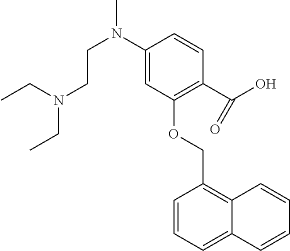 | 26.60 | 2.34 | 407 (MH⁺) |
| 15 ba) | 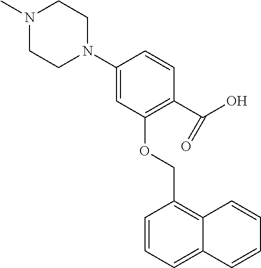 | 38.65 | 1.36 | 377 (MH⁺) |

### Example 16

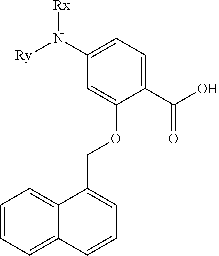

Rx, Ry=independently of each other as deducible from the individual compounds below.

Generic name: 4-Amino-2-(naphthalen-1-ylmethoxy)-benzoic acid derivatives

Generic Procedure:

### Parallel synthesis of 4-Amino-2-(naphthalen-1-ylmethoxy)-benzoic acids

To an array of glass tubes, one of 3 amines/anilines (0.400 mmol) is added into each of 3 tubes. Then a solution of 4-bromo-2-(naphthalen-1-ylmethoxy)-benzoic acid (0.267 mmol) in 1,2-dimethoxyethane (1.5 ml) and K₃PO₄ (0.800 mmol) is added to each tube. All tubes are flushed with argon and closed. Under argon atmosphere, 2-dicyclohexylphosphino-2′-(N,N-dimethylamino)biphenyl (0.0267 mmol) and tris(dibenzylideneacetone)dipalladium(0) (0.0267 mmol) are added to each tube. The resulting magnetically stirred mixtures are heated under a positive argon atmosphere at 90° C. for 17 hours. After cooling to room temperature, the reaction mixtures are individually transferred to 100 ml glass tubes, diluted with water, extracted several times with ethyl acetate, and the combined organic extracts are individually evaporated. Methanol (0.75 ml), tetrahydrofuran (0.75 ml) and a 2 molar aqueous solution of LiOH (0.67 ml) are individually added to the extracts and all tubes are closed. The array of reaction mixtures is allowed to stand at 80° C. for 17 hours. After cooling to room temperature acetic acid (0.058 ml) and tetrahydrofuran (2 ml) are individually added to the tubes, followed by filtration of each reaction mixture over a 0.45 μm PTFA membrane. The filtrates are then individually purified by a preparative LC-MS procedure.

Preparative LC-MS Procedure:

Preparative Waters chromatographic system with Micromass® ZQ MS detection. Aqueous acetonitrile of the following composition containing 0.1% of trifluoroacetic acid is used as a mobile phase at a flow rate of 50 ml/min using a Waters Sunfire® C-18 column (150×30 mm, 5 μm particle size): isocratic elution for 1 min at 10% aqueous acetonitrile followed by a linear gradient of 1.5 min from 10% aqueous acetonitrile to 60% aqueous acetonitrile followed by a linear gradient of 7.5 min from 60% aqueous acetonitrile to 95% aqueous acetonitrile followed by a linear gradient of 1.0 minute from 95% aqueous acetonitrile to 100% acetonitrile. The collection of products is triggered by the MS signal.

This generic procedure is used to prepare the following compounds:

| | | IC₅₀ | | Detected |
|---|---|---|---|---|
| Example | Formula | [umol I-1] | Rt [min] | mass |
| 16 a) | 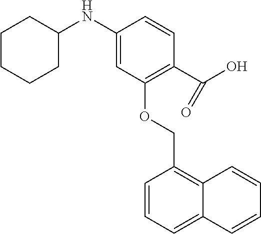 | | 1.91 | 376 (MH⁺) |
| 16 b) | 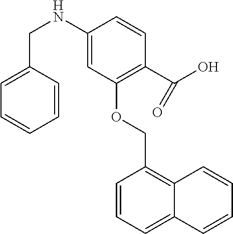 | 0.21 | 1.84 | 384 (MH⁺) |
| 16 c) | 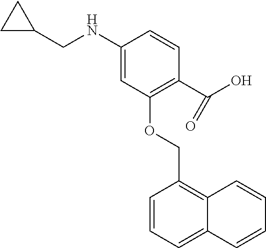 | 1.2 | 1.81 | 348 (MH⁺) |

### Example 17

Similar to Example 11 the following compounds are obtained by reaction of the corresponding salicylic acid derivative with the appropriate alkylating agent:

| Example | Formula | | |
|---|---|---|---|
| 17 a) | 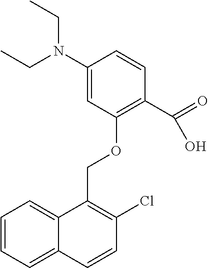 | | |
| 17 b) | 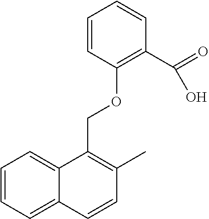 | | |
| 17 c) | 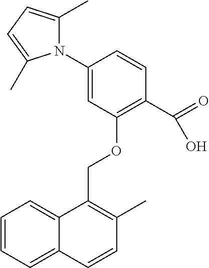 | | |
| 17 d) | 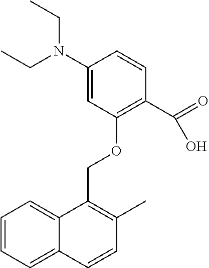 | | |
| 17 e) | 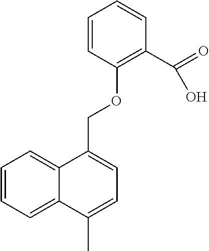 | | |
| 17 f) | 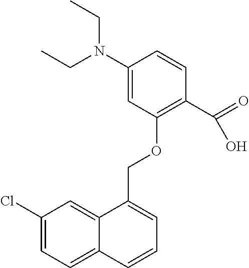 | | |
| 17 g) | 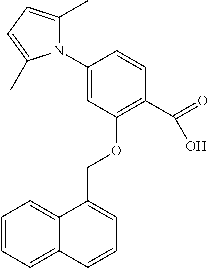 | | |
| 17 h) | 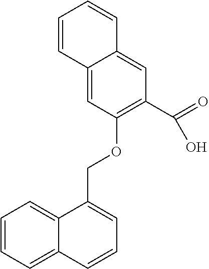 | | |
| 17 i) | 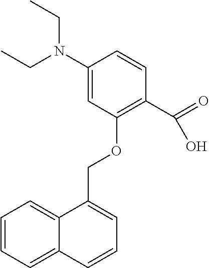 | | |
| 17 j) | 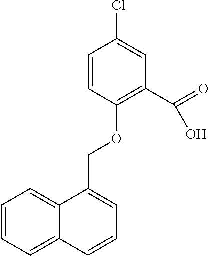 | | |
| 17 k) | 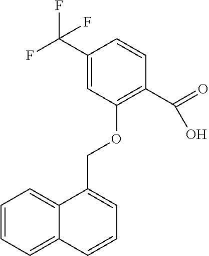 | | |
| 17 l) | 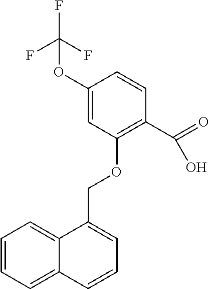 | | |
| 17 m) | 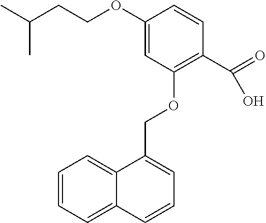 | | |
| 17 n) | 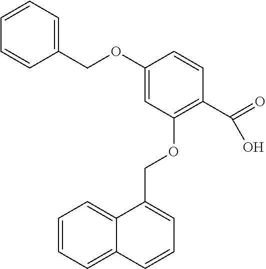 | | |
| 17 o) | 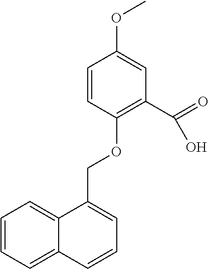 | | |
| 17 p) | 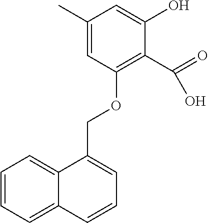 | | |
| 17 q) | 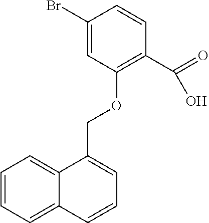 | | |
| 17 r) | 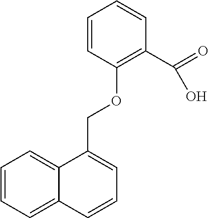 | | |
| 17 s) | 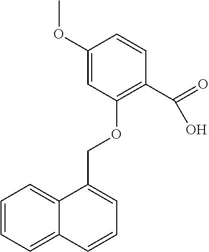 | | |
| Example | t_{Ret} [min] | LC Method | ES-MS |
| 17 a) | 1.87 | B | 384.0 [M + H]⁺ |
| 17 b) | 1.72 | B | 291.3 [M − H]⁻ |
| 17 c) | 1.9 | B | 386.2 [M + H]⁺ |
| 17 d) | 1.82 | B | 364.1 [M + H]⁺ |
| 17 e) | 1.72 | B | 291.3 [M − H]⁻ |
| 17 f) | 1.82 | B | 384.0 [M + H]⁺ |
| 17 g) | 1.83 | B | 372.4 [M + H]⁺ |
| 17 h) | 1.76 | B | 327.3 [M − H]⁻ |
| 17 i) | 1.78 | B | 348.2 [M − H]⁻ |
| 17 j) | 1.72 | B | 311.3 [M − H]⁻ |
| 17 k) | 1.75 | B | 345.3 [M − H]⁻ |
| 17 1) | 1.78 | B | 361.2 [M − H]⁻ |
| 17 m) | 1.91 | B | 363.4 [M − H]⁻ |
| 17 n) | 1.84 | B | 383.4 [M − H]⁻ |
| 17 o) | 1.65 | B | 307.3 [M − H]⁻ |
| 17 q) | 2.67 | A | |

### Example 18

### IC₅₀ on FPPS

The compound of Example 17r) has the following biological properties in the test system termed “FPS SPA” described above: IC₅₀=12.48 μM.

### Example 19

### Dry-Filled Capsules

5000 capsules, each comprising as active ingredient 0.25 g of one of the compounds of formula I mentioned in the preceding Examples, are prepared as follows:

Composition

| | |
|---|---|
| active ingredient | 1250 g |
| talcum | 180 g |
| wheat starch | 120 g |
| magnesium stearate | 80 g |
| lactose | 20 g |

Preparation process: The mentioned substances are pulverised and forced through a sieve of 0.6 mm mesh size. 0.33 g portions of the mixture are introduced into gelatin capsules using a capsule-filling machine.

### Example 20

### Soft Capsules

5000 soft gelatin capsules, each comprising as active ingredient 0.05 g of one of the compounds of formula I mentioned in the preceding Examples, are prepared as follows:

Composition

| | | |
|---|---|---|
| active ingredient | 250 | g |
| PEG 400 | 1 | litre |
| Tween 80 | 1 | litre |

Preparation process: The active ingredient is pulverised and suspended in PEG 400 (polyethylene glycol having an Mᵣ of from approx. 380 to approx. 420, Fluka, Switzerland) and Tween®80 (polyoxyethylene sorbitan monolaurate, Atlas Chem. Ind. Inc., USA, supplied by Fluka, Switzerland) and ground in a wet pulveriser to a particle size of approx. from 1 to 3 μm. 0.43 g portions of the mixture are then introduced into soft gelatin capsules using a capsule-filling machine.

### Example 21

### Inhibition According to the FPPS SPA

| Compound of Example | IC₅₀ (μmol/l) |
|---|---|
| 17 a) | 0.77 |
| 17 b) | 0.34 |
| 17 c) | 0.48 |
| 17 d) | 0.66 |
| 17 e) | 1.83 |
| 17 f) | 1.82 |
| 17 g) | 0.24 |
| 17 h) | 0.84 |
| 17 i) | 0.40 |
| 17 j) | 2.96 |
| 17 k) | 0.81 |
| 17 l) | 2.91 |
| 17 m) | 1.90 |
| 17 n) | 0.73 |
| 17 o) | 0.88 |
| 17 p) | 2.50 |
| 17 q) | 6.75 |
| 17 r) | 12.78 |
| 17 s) | 1.345 |

1. A compound of the formula I, 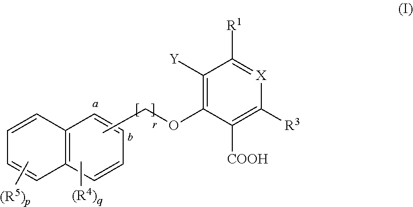
wherein R¹ is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, halo, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano;
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano;
p is an integer from 0, 1 to 4,
q is an integer from 0 to 3, and
r is 1 or 2,
or a pharmaceutically acceptable salt thereof,
for use in the treatment of a farnesyl pyrophosaphate synthase (FPPS) dependent disorder in a warm-blooded animal,

2. The compound of claim 1 wherein the warm-blooded animal is a human.

3. The compound of claim 1 of the formula IB; 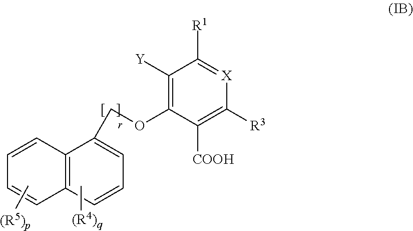
wherein
R¹ is hydrogen, unsubstituted alkyl, unsubstituted or substituted aryl, unsubstituted or substituted heterocyclyl, unsubstituted or substituted cycloalkyl, halo, —OR or —NR₂ wherein
R is, independently, of one another if present twice, hydrogen, unsubstituted or substituted alkyl, unsubstituted or substituted cycloalkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or halo,
or X is CR² and R¹ and R² together form a bridge of the formula #CH═CH—CH═C#H bound at the carbons marked with #, thus completing an annealed benzo group,
with R² preferably being hydrogen;
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
p is an integer from 0 to 4,
q is an integer from 0 to 3, and
r is 1 or 2,
with the proviso that at least one of R¹, R² and R³ must have a meaning mentioned for the present embodiment other than hydrogen;
or a pharmaceutically acceptable salt thereof.

4. The compound of claim 3, wherein at least one of p and q is one, or a pharmaceutically acceptable salt thereof.

5. The compound of claim 1, wherein
R¹ is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted cycloalkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
p is an integer from 0 to 4,
q is an integer from 0 to 3, and
r is 1 or 2,
with the proviso (i) that if R¹ is hydrogen, X is C—R² wherein R² is hydrogen, R³ is hydrogen, n is 1, q is 0 and p is 1, then R⁵ is substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, carboxy, acyl, nitro or cyano (that is, not unsubstituted alkyl); and with the proviso (ii) that if R¹ has one of the meanings defined above for this embodiment/claim other than hydrogen, and X, Y, R², R³, R⁴, n, q and r are as defined in this embodiment/claim before the provisos (i) and (ii), then p can also be 0 (zero) (that is only hydrogen is present, not a substituent R⁵) or p can also be 1 and R⁵ can also be unsubstituted alkyl;
or a pharmaceutically acceptable salt thereof.

6. The compound of claim 1, wherein
R¹ is unsubstituted alkyl; substituted alkyl selected from the group consisting of amino-C₁-C₇ alkyl, N-mono- or N,N-di-(C₁-C₇-alkyl, phenyl, C₁-C₇-alkanoyl and/or phenyl-lower alkyl)amino-C₁-C₇-alkyl and halo-C₁-C₇-alkyl; unsubstituted or substituted alkenyl, unsubstituted or substituted alkynyl, unsubstituted or substituted aryl, unsubstituted or substituted heterocyclyl, unsubstituted or substituted cycloalkyl, halo, —OR or —NR₂ wherein
R is, independently of one another if present twice, hydrogen, unsubstituted or substituted alkyl, unsubstituted or substituted cycloalkyl, unsubstituted or substituted alkanoyl, unsubstituted or substituted aryl, unsubstituted or substituted aroyl, unsubstituted or substituted heterocyclyl or unsubstituted or substituted heterocyclylcarbonyl (heterocyclyl-C(═O)—),
X is CR² wherein R² is hydrogen or, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen, hydroxyl, etherified hydroxyl or esterified hydroxyl;
each R⁴ if present, in the case that more than one moiety R⁴ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino, nitro or cyano,
each R⁵ if present, in the case that more than one moiety R⁵ is present independently of the others, is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, hydroxyl, etherified or esterified hydroxy, halo, amino, mono- or disubstituted amino,
p is an integer from 0 to 4,
q is an integer from 0 to 3, and
r is 1 or 2,
or a pharmaceutically acceptable salt thereof.

7. The compound of claim 1, wherein
R¹ is unsubstituted alkyl; substituted alkyl selected from the group consisting of amino-C₁-C₇-alkyl, N-mono- or N,N-di-(C₁-C₇-alkyl, phenyl, C₁-C₇-alkanoyl and/or phenyl-lower alkyl)amino-C₁-C₇-alkyl and halo-C₁-C₇-alkyl; unsubstituted or substituted aryl, unsubstituted or substituted heterocyclyl, halo, —OR or —NR₂ wherein
R is, independently of one another if present twice, hydrogen, unsubstituted or substituted alkyl, unsubstituted or substituted cycloalkyl, unsubstituted or substituted aryl or unsubstituted or substituted heterocyclyl,
X is CR² wherein R² is hydrogen, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl;
each R⁴ if present, in the case that more than one moiety R⁴ is present independently of the others, is unsubstituted or substituted alkyl, hydroxyl, etherified hydroxyl, halo or cyano,
each R⁵ if present, in the case that more than one moiety R⁵ is present independently of the others, is unsubstituted or substituted alkyl, hydroxyl, etherified hydroxyl, halo or cyano,
p is an integer from 0 to 4,
q is an integer from 0 to 3, and
r is 1 or 2,
or a pharmaceutically acceptable salt thereof.

8. The compound of claim 1, wherein
R¹ is hydrogen, C₁-C₇-alkyl, amino-C₁-C₇-alkyl, N-mono- or N,N-di-(C₁-C₇-alkyl, phenyl, C₁-C₇-alkanoyl and/or phenyl-lower alkyl)-amino-C₁-C₇-alkyl, halo-C₁-C₇-alkyl (e.g. trifluoromethyl), halo, C₁-C₇-alkoxy, hydroxy-C₁-C₇-alkoxy, carboxy-C₁-C₇-alkoxy, halo-C₁-C₇-alkoxy, phenyl- or naphthyl-C₁-C₇-alkoxy, amino, N-mono- or N,N-di-{C₁-C₇-alkyl, hydroxy-C₁-C₇-alkyl, C₁-C₇-alkoxy-C₁-C₇-alkyl, [N′,N′-di-(C₁-C₇-alkyl)-amino-C₁-C₇-alkyl, C₃-C₈-cycloalkyl, mono- to tri-[C₁-C₇-alkyl and/or hydroxy]-C₃-C₈-cycloalkyl, phenyl, naphthyl, mono- to tri-[C₁-C₇-alkyl, halo and/or cyano]-phenyl, mono- to tri-[C₁-C₇-alkyl, halo and/or cyano]-naphthyl, C₁-C₇-alkanoyl, phenyl-C₁-C₇-alkyl, phenyl-C₁-C₇-alkyl, C₃-C₈-cycloalkyl-C₁-C₇-alkyl, [(C₁-C₇-alkyl)-C₃-C₈-cycloalkyl]-C₁-C₇-alkyl, [hydroxy-C₃-C₈-cycloalkyl]-C₁-C₇-alkyl, [C₁-C₇-alkyl-pyrrolidinyl]-C₁-C₇-alkyl, [tetrahydrofuranyl-C₁-C₇-alkyl, thiophenyl-C₁-C₇-alkyl, pyridinyl-C₁-C₇-alkyl, C₁-C₇-alkylpyrazolidinyl, pyridinyl and/or C₁-C₇-alkylpiperidinyl}-amino, (or especially) phenyl, naphthyl, mono-, di- or tri-(C₁-C₇-alkyl)-phenyl or -naphthyl, hydroxyl-C₁-C₇-alkyl-phenyl or -naphthyl, mono-, di- or tri-(halo)-phenyl or -naphthyl, hydroxyphenyl, hydroxynaphthyl, mono-, di- or tri-(C₁-C₇-alkoxy)-phenyl or -naphthyl, halo-C₁-C₇-alkoxyphenyl or -naphthyl (e.g. trifluoromethoxyphenyl), C₁-C₇-alkanoyl-phenyl or -naphthyl, azido-C₁-C₇-alkylphenyl, amino-C₁-C₇-alkylphenyl, benzo[1,3]dioxolyl, 2,3-dihydro-benzo[1,4]dioxinyl, pyrrolyl, 2,5-di-(C₁-C₇-alkyl)pyrrolyl, pyrrolidinyl, oxopyrrolidinyl, mono- or di-C₁-C₇-alkylpyrrolidinyl, furanyl, piperidinyl, C₁-C₇-alkoxypyridinyl, hydroxy-C₁-C₇-alkylpiperidinyl (especially -piperidino), piperazinyl, especially piperazino, C₁-C₇-alkylpiperazinyl, especially -piperazino, C₁-C₇-alkyl-piperazinyl, especially -piperazinyl, morpholinyl, especially morpholino, thiomorpholinyl, especially thiomorpholino, S-oxo-thiomorpholinyl, especially S-oxo-thiomorpholino, S,S-dioxothiomorpholinyl, especially S,S-dioxo-thiomorpholino, azepanyl, especially azepan-1-yl, C₁-C₇-alkyl-1,4-diazepanyl, especially -diazepan-1-yl, indolyl, indolyl, N-(C₁-C₇-alkyl)-indolyl, benzofuranyl or benzothiophenyl,
X is CR² wherein R² is hydrogen or, if the napthyl ring is bound to the rest of the molecule in formula I via its carbon marked “a”, hydrogen or halo, preferably hydrogen;
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl,
each R⁴ and/or R⁵ if present, in the case that more than one moiety R⁴ and/or R⁵ is present independently of the others, is C₁-C₇-alkyl, hydroxy-C₁-C₇-alkyl, hydroxy, tri-(C₁-C₇-alkylsilyl)-C₁-C₇-alkoxy-C₁-C₇-alkoxy, halo, C₁-C₇-alkoxycarbonyl or cyano;
p is 0, 1 or 2,
q is 0, 1 or 2 and
r is 1 or 2, preferably 1,
or a pharmaceutically acceptable salt thereof.

9. The compound of claim 8, wherein
R¹ is C₁-C₇-alkyl, amino-C₁-C₇-alkyl, N-mono- or N,N-di-(C₁-C₇-alkyl, phenyl, C₁-C₇-alkanoyl and/or phenyl-lower alkyl)-amino-C₁-C₇-alkyl, halo-C₁-C₇-alkyl, halo, C₁-C₇-alkoxy, hydroxy-C₁-C₇-alkoxy, carboxy-C₁-C₇-alkoxy, halo-C₁-C₇-alkoxy, phenyl- or naphthyl-C₁-C₇-alkoxy, amino, N-mono- or N,N-di-{C₁-C₇-alkyl, hydroxy-C₁-C₇-alkyl, C₁-C₇-alkoxy-C₁-C₇-alkyl, [N′,N′-di-(C₁-C₇-alkyl)-amino-C₁-C₇-alkyl, C₃-C₈-cycloalkyl, mono- to tri-[C₁-C₇-alkyl and/or hydroxy]-C₃-C₈-cycloalkyl, phenyl, naphthyl, mono- to tri-[C₁-C₇-alkyl, halo and/or cyano]-phenyl, mono- to tri-[C₁-C₇-alkyl, halo and/or cyano]-naphthyl, C₁-C₇-alkanoyl, phenyl-C₁-C₇-alkyl, phenyl-C₁-C₇-alkyl, C₃-C₈-cycloalkyl-C₁-C₇-alkyl, [(C₁-C₇-alkyl)-C₃-C₈-cycloalkyl]-C₁-C₇-alkyl, [hydroxy-C₃-C₈-cycloalkyl]-C₁-C₇-alkyl, [C₁-C₇-alkyl-pyrrolidinyl]-C₁-C₇-alkyl, [tetrahydrofuranyl-C₁-C₇-alkyl, thiophenyl-C₁-C₇-alkyl, pyridinyl-C₁-C₇-alkyl, C₁-C₇-alkylpyrazolidinyl, pyridinyl and/or C₁-C₇-alkylpiperidinyl}-amino, (or especially) phenyl, naphthyl, mono-, di- or tri-(C₁-C₇-alkyl)-phenyl or -naphthyl, hydroxyl-C₁-C₇-alkyl-phenyl or naphthyl, mono-, di- or tri-(halo)-phenyl or -naphthyl, hydroxyphenyl, hydroxynaphthyl, mono-, di- or tri-(C₁-C₇-alkoxy)-phenyl or -naphthyl, halo-C₁-C₇-alkoxy-phenyl or -naphthyl (e.g. trifluoromethoxyphenyl), C₁-C₇-alkanoyl-phenyl or -naphthyl, azido-C₁-C₇-alkylphenyl, amino-C₁-C₇-alkylphenyl, benzo[1,3]dioxolyl, 2,3-dihydro-benzo[1,4]dioxinyl, pyrrolyl, 2,5-di-(C₁-C₇-alkyl)pyrrolyl, pyrrolidinyl, oxopyrrolidinyl, mono- or di-C₁-C₇-alkylpyrrolidinyl, furanyl, piperidinyl, C₁-C₇-alkoxypyridinyl, hydroxy-C₁-C₇-alkylpiperidinyl (especially -piperidino), piperazinyl, especially piperazino, C₁-C₇-alkylpiperazinyl, especially -piperazino, C₁-C₇-alkylpiperazinyl, especially -piperazinyl, morpholinyl, especially morpholino, thiomorpholinyl, especially thiomorpholino, S-oxo-thiomorpholinyl, especially S-oxo-thiomorpholino, S,S-dioxothiomorpholinyl, especially S,S-dioxo-thiomorpholino, azepanyl, especially azepan-1-yl, C₁-C₇-alkyl-1,4-diazepanyl, especially -diazepan-1-yl, indolyl, indolyl, N-(C₁-C₇-alkyl)-indolyl, benzofuranyl or benzothiophenyl,
X is CR² wherein R² is hydrogen, if the napthyl ring is bound to the rest of the molecule in formula I via its carbon marked “a”, hydrogen or halo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl,
each R⁴ and/or R⁵ if present, in the case that more than one moiety R⁴ and/or R⁵ is present independently of the others, is C₁-C₇-alkyl, hydroxy-C₁-C₇-alkyl, hydroxy, halo, C₁-C₇-alkoxycarbonyl, cyano or tri-(C₁-C₇-alkylsilyl)-C₁-C₇-alkoxy-C₁-C₇-alkoxy;
p is 0, 1 or 2,
q is 0, 1 or 2 and
r is 1 or 2, preferably 1,
or a pharmaceutically acceptable salt thereof.

10. The compound of claim 1, wherein
R¹ is methyl, aminomethyl, trifluoromethyl, phenyl, 2-methylphenyl, 3-methylphenyl, 2,4-dimethyl-phenyl, 2,6-dimethylphenyl, 3-(hydroxymethyl)phenyl, 4-(hydroxymethyl)-phenyl, 2-fluorophenyl, 3-fluorophenyl, 4-fluorophenyl, 3,4-difluoro-phenyl, 4-chlorophenyl, 3,4-dichlorophenyl, 3-hydroxyphenyl, 4-hydroxyphenyl, 2-methoxyphenyl, 3-methoxyphenyl, 2,6-dimethoxy-phenyl, 3,4-dimethoxyphenyl, 3-trifluoromethoxyphenyl, 4-acetylaminophenyl, 3-formylphenyl, 3-azidomethylphenyl, 3-aminomethylphenyl, benzo[1,3]dioxol-5-yl, 2,3-dihydro-benzo[1,4]dioxin-6-yl, pyrrol-1-yl, 2,5-dimethyl-pyrrol-1-yl, pyrrolidino, 2-oxopyrrolidino, 2,5-dimethylpyrrolidino, furan-3-yl, piperidino, 3-hydroxymethylpiperidino, piperazino, 4-methylpiperazino, 4-acetyl-piperazino, morpholino, 2-methoxy-pyridin-3-yl, azepan-1-yl, 4-methyl-1,4-diazepan-1-yl, indol-5-yl, indol-4-yl, N-methyl-indol-5-yl, 1-benzofuran-2-yl, 1-benzothiophen-3-yl, bromo, chloro, methoxy, 3-methyl-n-butoxy, 2-hydroxy-ethoxy, carboxymethyloxy, trifluoromethoxy, benzyloxy, N-methylamino, N-ethylamino, N,N-dimethylamino, N,N-diethylamino, N-(n-propyl)-amino, N-(2,2-dimethylpropyl)-amino, N-(1,2,2-trimethylpropyl)-amino, N-(1-(ethyl)-n-propyl)-amino, N-(2-hydroxyethyl)-amino, N-(3-hydroxypropyl)-amino, N-(2-methoxyethyl)-amino, N-(3-methoxypropyl)-amino, N-(2-methoxy-1-methyl-ethyl)-amino*, N-(2-methoxyethyl)-N-methyl-amino, N-(2-hydroxyethyl)-N-methyl-amino, N-benzylamino, N-cyclopropylmethyl-amino, N-cyclohexylmethyl-amino, N-(1-cyclohexyl-ethan-1-yl)-amino*, N-cyclopropylmethyl-N-(n-propyl)-amino, N-[2-(N′,N′-diethylamino)-ethyl]-N-methyl-amino, N-phenylamino, N-(2-methylphenyl-amino, N-(4-methylphenyl)-amino, N-(2,6-dimethylphenyl)-amino, N-(naphthalin-2-yl)-amino, N-(4-isopropylphenyl)-amino, N-(2-fluorophenyl)-amino, N-(2-chlorophenyl)-amino, N-(3-chlorophenyl)-amino, N-(2-cyanophenyl)-amino, N-(3-chlorphenyl)-N-methyl-amino, N-(cyclobutyl)-amino, N-(cyclopentyl)-amino, N-(cycloheptyl)-amino, N-(4-methyl-cyclohexyl)amino*, N-(4-hydroxycyclohexyl)-amino,* N-[3-(1-methyl-pyrrolidin-2-yl)-propyl]-amino, N-(tetrahydrofuran-2-ylmethyl)-amino, N-(thiophen-2-ylmethyl)-amino, N-[2-(pyridin-2-yl)-ethyl]-N-methyl-amino, N-(1-methylpyrazolidin-5-yl)-amino, N-(pyridin-2-yl)-amino, N-(pyridin-3-yl)amino, N-(pyridin-4-yl)amino or N-(1-methylpiperidin-4-yl)-amino (where the moieties with an asterisk (*) can preferably be present in a form where each chiral carbon is present in isomerically pure form, that is, as R- or S-form);
X is CR² wherein R² is hydrogen or, if the napthyl ring is bound to the rest of the molecule in formula I via its carbon marked “a”, hydrogen, chloro or bromo,
or X is CR² and R¹ and R² together form a bridge of the formula #CH═CH—CH═C#H bound at the carbons marked with #, thus completing an annealed benzo group,
Y is hydrogen or together with R¹ forms a bridge of the formula #CH═CH—CH═C#H bound at the carbons marked with #, thus completing an annealed benzo group,
with the proviso that not more than one of the pairs R¹ and R² or Y and R¹ form a bridge as defined above;
R³ is hydrogen or hydroxyl,
each R⁴ and/or R⁵ if present, in the case that more than one moiety R⁴ and/or R⁵ is present independently of the others, is methyl, hydroxymethyl, hydroxyl, 3-(2-trimethylsilyl-ethoxymethoxy, chloro, methoxycarbonyl or cyano;
p is 0 or 1,
q is 0 or 1 and
r is 1,
or a pharmaceutically acceptable salt thereof.

11. The compound of claim 1, selected from the group consisting of
4-(2-methyl-phenyl)-2-(naphthalin-1-ylmethoxy)-benzoic acid,
4-phenyl-2-(naphthalin-1-ylmethoxy)-benzoic acid,
4-(2,6-dimethoxy-phenyl)-2-(naphthalin-1-ylmethoxy)-benzoic acid,
4-(3-aminomethyl-phenyl)-2-(naphthalin-1-ylmethoxy)-benzoic acid,
4-(2-oxopyrrolidino)-2-(naphthalin-1-ylmethoxy)-benzoic acid,
4-pyrrolo-2-2-(naphthalin-1-ylmethoxy)-benzoic acid,
4-piperidino-2-(naphthalin-1-ylmethoxy)-benzoic acid,
4-phenylamino-2-(naphthalin-1-ylmethoxy)-benzoic acid,
4-(4-carbamoyl-phenyl)-2-(naphthalin-1-ylmethoxy)-benzoic acid,
2-(5-cyano-napthalin-1-yl-methoxy)-benzoic acid,
4-(2-hydroxyethoxy)-2-(naphthalin-1-ylmethoxy)-benzoic acid,
4-carboxymethoxy-2-(naphthalin-1-ylmethoxy)-benzoic acid
or a pharmaceutically acceptable salt thereof.

12. The compound of claim 1, selected from 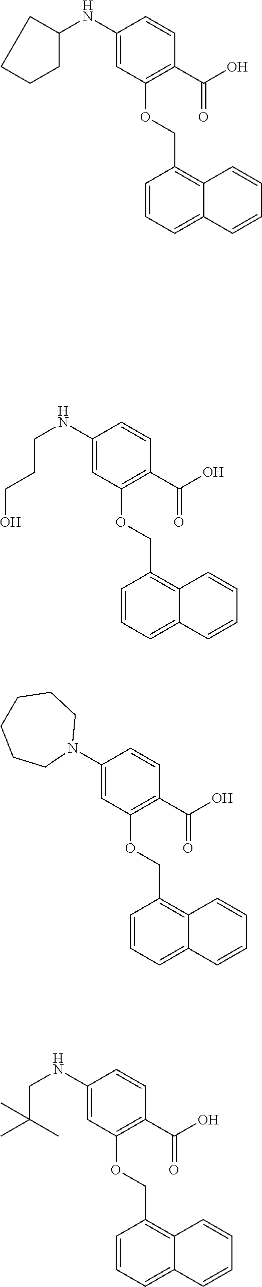 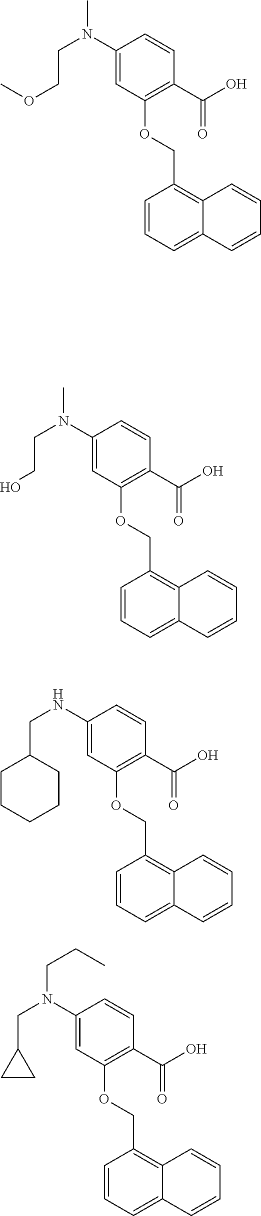 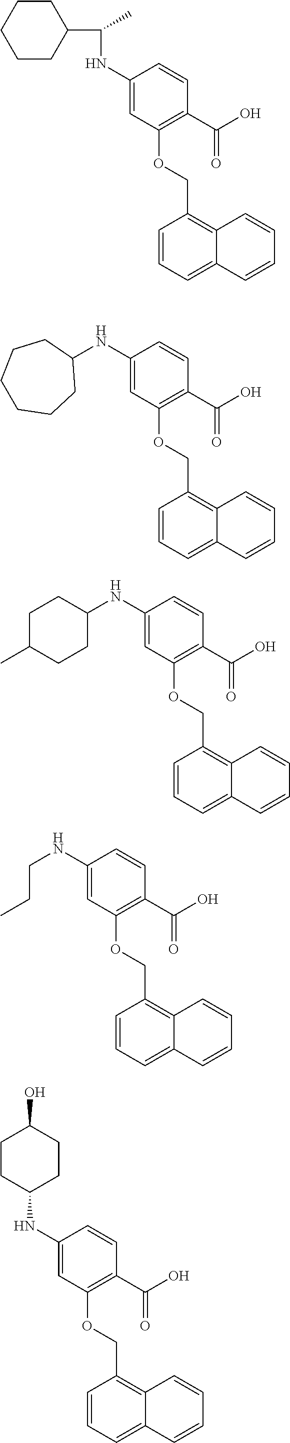 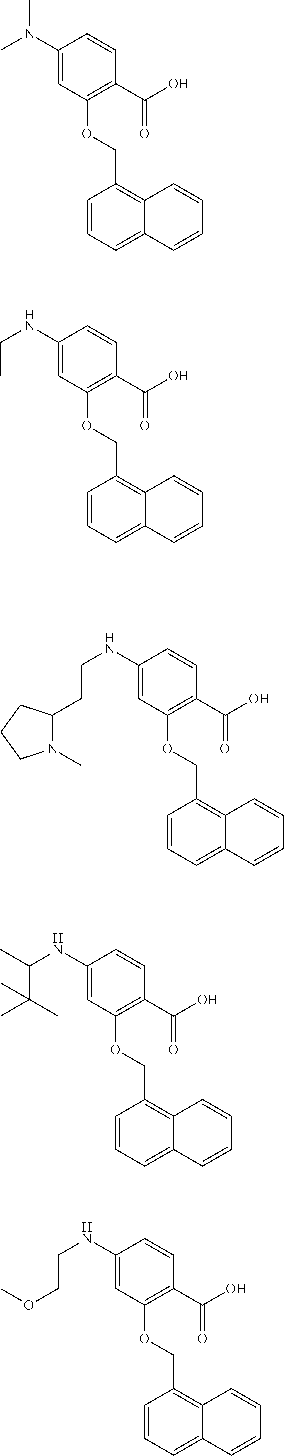 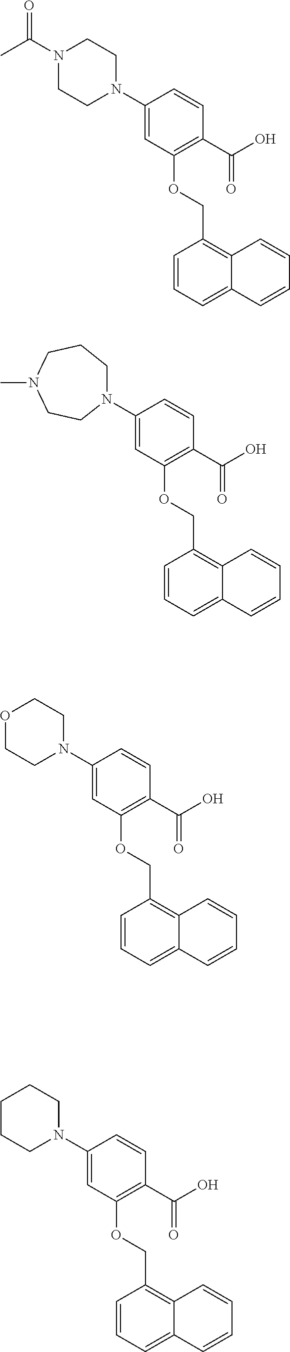 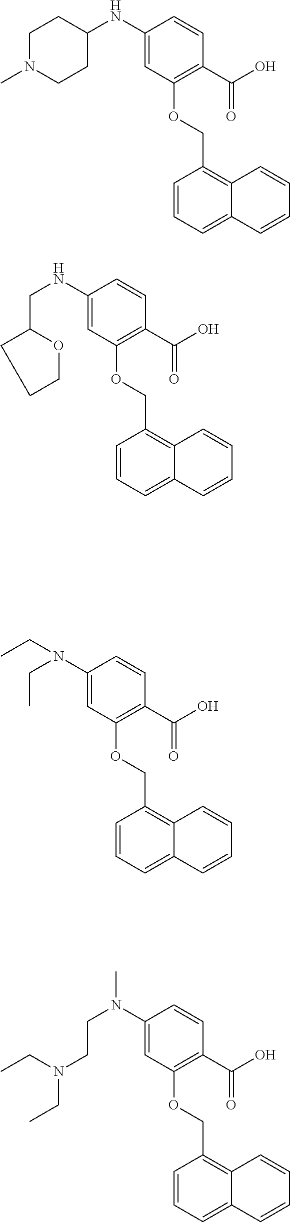 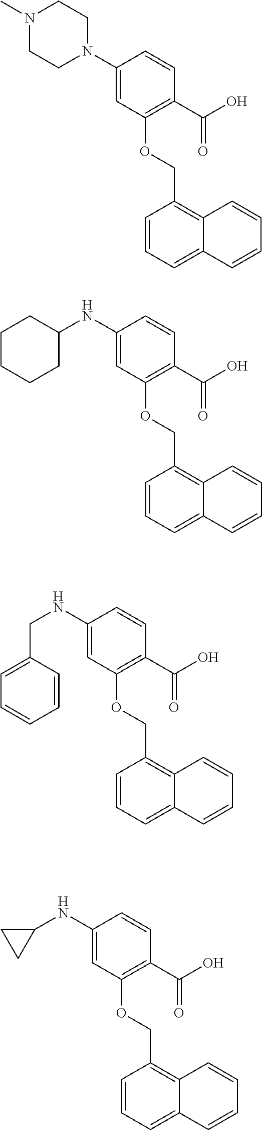 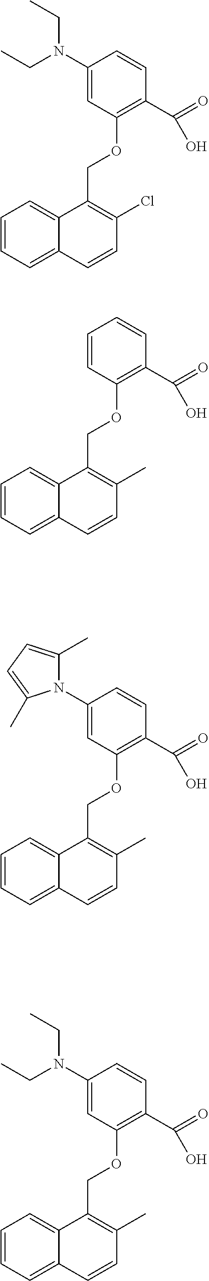 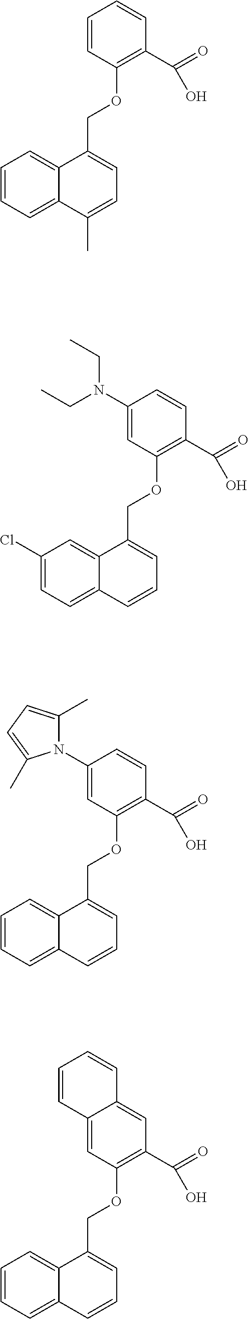 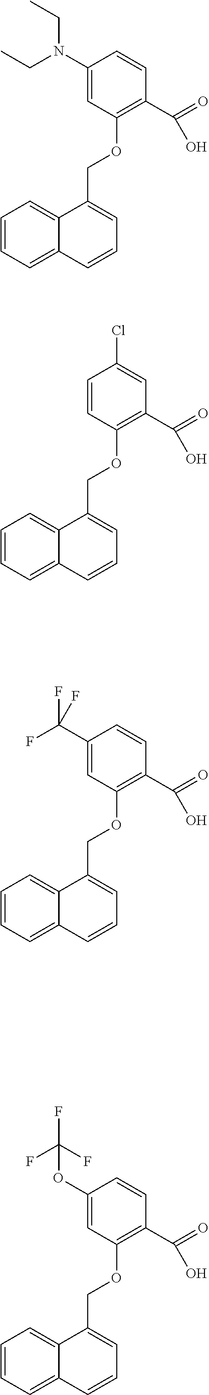 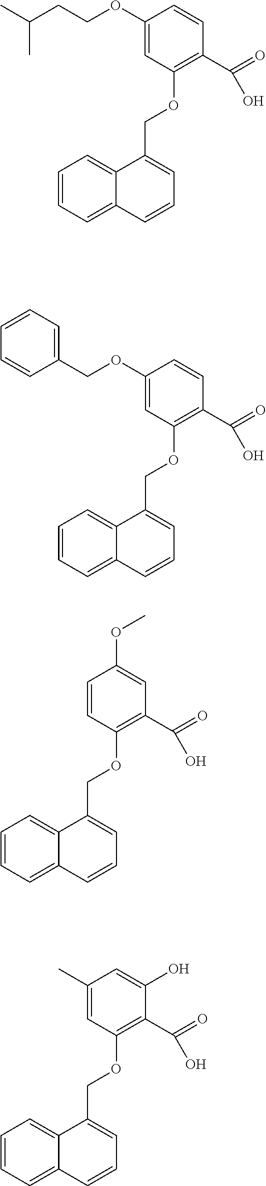 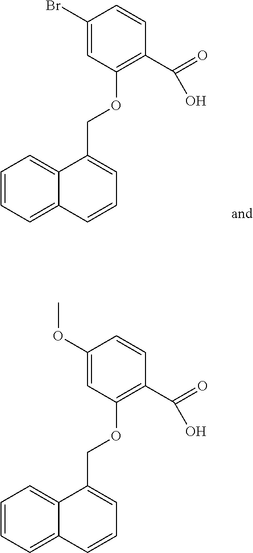
or a pharmaceutically acceptable salt thereof.

13. The compound according to claim 1 with the formula 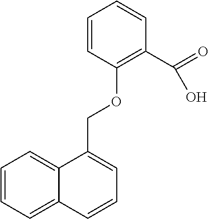
or a pharmaceutically acceptable salt thereof, for use in the treatment of an FPPS-dependent disease.

14. A pharmaceutical preparation, comprising the compound of claim 1, or a pharmaceutically acceptable salt thereof, and at least one pharmaceutically acceptable carrier material.

15. (canceled)

16. A method of treatment of a FPPS-dependent disease, comprising administering the compound of claim 1, or a pharmaceutically acceptable salt thereof, in a therapeutically effective amount to a warm-blooded animal, where in need of such treatment.

17. A method of preparing a pharmaceutical preparation for the treatment of a FPPS dependent disease, comprising mixing the compound of claim 1, or a pharmaceutically acceptable salt thereof, with at least one pharmaceutically acceptable carrier material.

18. The method of claim 16 wherein the warm-blooded animal is a human.
